(19) United States
(12) Patent Application Publication
(10) Pub. No.: US 2004/0220640 A1
(43) Pub. Date: Nov. 4, 2004

(54) Method and apparatus for determining myocardial electrical resitution and controlling extra systolic stimulation

(75) Inventors: John E. Burnes, Andover, MN (US); Vincent E. Splett, Apple Valley, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/426,979

(22) Filed: Apr. 29, 2003

(51) Int. Cl. A61N 1/37

(52) U.S. Cl. 607/28

(57) ABSTRACT

A system and method are provided for controlling extra systolic intervals during extra systolic stimulation delivered to effectively produce post-extra systolic potentiation (PESP) to improve hemodynamic function for the treatment of cardiac mechanical insufficiency. Controlling the interval is based on measurements of the electrical restitution properties of myocardial tissue. A parameter related to the action potential duration is measured from an electrical signal received from the heart during extra systolic stimulation at different intervals. An electrical restitution condition is determined from the measured action potential duration related parameter. An operating interval is set based on the measured electrical restitution. Methods for controlling the interval further include setting the operating ESI based on electrical restitution and/or the mechanical effect of PESP on post-extra systoles. Methods for controlling the interval include setting the operating interval based on a measure of electrical restitution and a measure of mechanical restitution.

### CROSS REFERENCE TO RELATED APPLICATIONS

This patent hereby incorporates by reference the following patent applications filed on even date hereof; namely, P-11030, “Cardiac Pacing Therapy Parameter Programming;” P-11214, “Method and Apparatus for Detecting Myocardial Electrical Recovery and Controlling Extra-Systolic Stimulation;” P-11216, “Method and Apparatus to Monitor Pulmonary Edema;” and P-11215, “Use of Activation and Recovery Times and Dispersions to Monitor Heart Failure Status and Arrhythmia Risk.”

### FIELD OF THE INVENTION

The present invention relates generally to the field of cardiac stimulation devices and more specifically to a device and method for delivering extra systolic stimulation to achieve post-extra systolic potentiation to improve hemodynamic function in the treatment of cardiac mechanical insufficiency. In particular, a device and method are provided for measuring myocardial electrical restitution and adjusting the timing of extra systolic stimulation based on the electrical restitution measurement.

### BACKGROUND OF THE INVENTION

Post-extra systolic potentiation (PESP) is a property of cardiac myocytes that results in enhanced mechanical function of the heart on the beats following an extra systolic stimulus delivered early after either an intrinsic or pacing-induced systole. The magnitude of the enhanced mechanical function is strongly dependent on the timing of the extra systole relative to the preceding intrinsic or paced systole. When correctly timed, an extra systolic stimulation pulse causes an electrical depolarization of the heart but the attendant mechanical contraction is absent or substantially weakened. The contractility of the subsequent cardiac cycles, referred to as the post-extra systolic beats, is increased as described in detail in commonly assigned U.S. Pat. No. 5,213,098 issued to Bennett et al., incorporated herein by reference in its entirety.

The mechanism of PESP is thought to be related to the calcium cycling within the myocytes. The extra systole initiates a limited calcium release from the sarcolasmic reticulum (SR). The limited amount of calcium that is released in response to the extra systole is not enough to cause a normal mechanical contraction of the heart. After the extra systole, the SR continues to take up calcium with the result that subsequent depolarization(s) cause a large release of calcium from the SR, resulting in vigorous myocyte contraction.

As noted, the degree of mechanical augmentation on post-extra systolic beats depends strongly on the timing of the extra systole following a first depolarization, referred to as the extrasystolic interval (ESI). If the ESI is too long, the PESP effects are not achieved because a normal mechanical contraction takes place in response to the extra systolic stimulus. As the ESI is shortened, a maximal effect is reached when the ESI is slightly longer than the physiologic refractory period. An electrical depolarization occurs without a mechanical contraction or with a substantially weakened contraction. When the ESI becomes too short, the stimulus falls within the absolute refractory period and no depolarization occurs.

The above-cited Bennett patent generally discloses a post-extra systolic potentiation stimulator for the treatment of congestive heart failure or other cardiac dysfunctions. A cardiac performance index is developed from a sensor employed to monitor the performance of the heart, and a cardiac stress index is developed from a sensor employed to monitor the cardiac muscle stress. Either or both the cardiac performance index and cardiac stress index may be used in controlling the delivery of PESP stimulation. PCT Publication WO 02/053026 issued to Deno et al., incorporated herein by reference in its entirety, discloses an implantable medical device for delivering post extra systolic potentiation stimulation. PESP stimulation is employed to strengthen the cardiac contraction when one or more parameters indicative of the state of heart failure show that the heart condition has progressed to benefit from increased contractility, decreased relaxation time, and increased cardiac output. PCT Publication WO 01/58518 issued to Darwish et al., incorporated herein by reference in its entirety, generally discloses an electrical cardiac stimulator for improving the performance of the heart by applying paired pulses to a plurality of ventricular sites. Multi-site paired pacing is proposed to increase stroke work without increasing oxygen consumption and, by synchronizing the timing of the electrical activity at a plurality of sites in the heart, decrease a likelihood of development of arrhythmia.

As indicated in the referenced '098 patent, one risk associated with PESP stimulation is arrhythmia induction. If the extrasystolic pulse is delivered to cardiac cells during the vulnerable period, the risk of inducing tachycardia or fibrillation in arrhythmia-prone patients is high. The vulnerable period encompasses the repolarization phase of the action potential, also referred to herein as the “recovery phase” and a period immediately following it. During the vulnerable period, the cardiac cell membrane is transiently hyper-excitable. Therefore, although the property of PESP has been known of for decades, the application of PESP in a cardiac stimulation therapy for improving the mechanical function of the heart has not been realized clinically because of the perceived risks.

Electrical restitution is the relationship between changes in action potential duration with varying diastolic intervals occurring between a first cardiac systole and an extra systole. Restitution reflects the recovery properties of the cardiac tissue with respect to the time of initiation of the extra systole. An electrical restitution curve can be constructed by measuring the action potential duration over a range of extra systolic intervals. The curve is initially very steep where short extra systolic intervals result in a greater shortening of the action potential durations. After the initially steep portion, a plateau is reached as the APD reaches a maximum at longer extra systolic intervals. The restitution curve measured in human ventricular myocardium has been found to have a biphasic “hump” prior to the plateau phase. The slope of the electrical restitution curve over the entire range of extra systolic intervals, referred to as R_{K}, or the slope of the steepest portion of the curve, referred to as Rₛ, can be used as a measure of the responsiveness of APD changes to a change in extra systolic interval.

During clinical electrophysiological studies, premature ventricular stimulation is applied to determine if ventricular arrhythmias are inducible, indicating a patient's propensity for arrhythmias. The shortened action potential duration resulting from the extra systole occurring at a shortened diastolic interval alters the refractoriness of the myocardium, which is believed to set up pathways for reentrant depolarizations. Increased dispersion of action potential duration and refractoriness is associated with an increase risk of arrhythmias. Others have reported that premature stimuli delivered at short intervals increase the dispersion of repolarization and the orientation of repolarization gradients over the ventricles in a way that greatly enhances susceptibility to fibrillation. Dispersion of electrical restitution, i.e. differences in the response of the action potential duration to changes in extra systolic intervals at different myocardial sites, can cause substantial changes in action potential duration dispersion and recovery dispersion in response to extra systoles. Therefore, premature stimuli can create a substrate for arrhythmias due to increased heterogeneity of refractoriness. Exaggerated action potential duration shortening after premature ventricular extra stimulation coupled with altered restitution characteristics in diseased myocardium may further contribute to the arrhythmogenic substrate.

In delivering extra systolic stimulation for achieving mechanical enhancement of cardiac function on post-extra systolic beats, therefore, it is important to avoid extra systolic intervals that produce exaggerated shortening of the action potential duration and increased dispersion of the action potential duration and refractoriness. When safely delivered, the mechanical effects of PESP may advantageously benefit a large number of patients suffering from cardiac mechanical insufficiency, such as patients in heart failure. Hence, a method for controlling the timing of the extra systolic stimuli during extra systolic stimulation is needed that avoids increased dispersion of refractoriness due to heightened action potential duration shortening.

### SUMMARY OF THE INVENTION

The present invention provides a system and method for controlling the extra systolic interval (ESI) during extra systolic stimulation therapy delivered to effectively produce post-extra systolic potentiation (PESP) for the treatment of cardiac mechanical insufficiency. In a preferred embodiment, the ESI is controlled based on measurements of the electrical restitution properties of the myocardial tissue. As such, the system includes an implantable medical device and associated lead system for delivering electrical stimulation pulses to the heart and receiving and processing electrical cardiac signals from the heart. The system may further include arrhythmia detection and therapy delivery capabilities. In some embodiments, the system further includes one or more physiological sensors for measuring cardiac hemodynamic or contractile function in order to assess the strength of the myocardial contraction during extra systoles and/or during post-extra systolic heart beats.

The method for controlling the ESI includes measuring a parameter related to myocyte action potential duration from an electrical signal received from the heart during intrinsic or stimulation-induced extra systoles occurring at a number of different ESIs. A measure of electrical restitution is determined from the measured extra systolic action potential duration related parameter. In a preferred embodiment, the action potential duration related parameter is the activation-recovery interval (ARI) measured from an EGM or subcutaneous ECG signal. An electrical restitution curve is constructed from ARIs measured during extra systoles occurring at a number of ESIs or diastolic intervals (DIs). The operating ESI during ESS is set according to a desired operating point on the electrical restitution curve. Preferably the operating point is located along the plateau portion of the electrical restitution curve or at the transition point between the steep phase associated with heightened action potential duration shortening and the plateau phase of the electrical restitution curve.

After setting an initial operating point for the ESI, adjustments of the ESI may be made based on: periodic measurements of an action potential duration related parameter at a shortened ESI; an abrupt change in the action potential duration related parameter measured during the extra systole on a beat-by-beat or other less frequent basis; a change in the relationship between the action potential duration related parameter measured during the primary systole and the action potential duration related parameter measured during the extra systole as measured on a beat-by-beat or less frequent basis; and/or a change in an index of electrical restitution which may be a slope or other measured feature of the electrical restitution curve.

In an alternative embodiment, a method for controlling the ESI includes setting the operating ESI based on a measure of electrical restitution and further includes adjusting the operating ESI within safe limits set according to the electrical restitution measurement in order to maximize the mechanical PESP effect on post-extra systoles. The mechanical PESP effect is determined from a physiological sensor capable of generating a signal proportional to myocardial contractile performance or cardiac hemodynamic performance or a metabolic state.

In yet another alternative embodiment, a method for controlling the ESI includes setting the operating ESI based on a measure of electrical restitution and/or a measure of mechanical restitution. Mechanical restitution is measured by determining the mechanical response to an extra systole occurring at a number of ESIs. The mechanical response is measured from a physiological sensor capable of generating a signal proportional to myocardial contractile performance. An optimal operating ESI preferably minimizes the myocardial mechanical response to the extra systolic stimulus in order to achieve a maximal mechanical PESP effect.

In yet another embodiment, a “look-up” table of ESIs is compiled by generating a family of electrical restituion curves or electrical restitution measurement parameters for varying heart rates. ESIs for a number of heart rate zones are stored as desired operating points on the corresponding restitution curves. During ESS therapy delivery, the ESI is adjusted according to “look-up” table values as heart rate or pacing rate varies.

In yet another embodiment, the control of ESS therapy includes monitoring for increases in the spatial dispersion of electrical restitution. If restitution dispersion is increased, an ESI is adjusted or ESS therapy may be aborted in order to avoid increased risk of arrhythmias.

The present invention advantageously allows extra systolic stimulation to be safely delivered such that the benefits of enhanced mechanical function due to PESP may be realized in clinical treatments for cardiac mechanical insufficiency without increasing the risk of arrhythmias.

### BRIEF DESCRIPTION OF THE DRAWINGS

FIG. 1A is an illustration of an exemplary implantable medical device (IMD) in which the present invention may be implemented.

FIG. 1B is an illustration of an alternative IMD including subcutaneous ECG electrodes incorporated in the housing of the IMD.

FIG. 2A is a functional schematic diagram of the implantable medical device shown in FIG. 1A.

FIG. 2B is a functional schematic diagram of an alternative embodiment of the IMD, with regard to the electrode configuration of FIG. 1B, which includes dedicated circuitry for measuring electrical restitution.

FIG. 3 is a flow chart providing an overview of methods included in one embodiment of the present invention for controlling extra systolic stimulation based on measurements of electrical restitution.

FIG. 4A depicts a representative unipolar EGM signal illustrating one method for measuring the activation recovery interval, which may be employed by the method of FIG. 3 in collecting electrical restitution data.

FIG. 4B is a timing diagram shown in temporal relation to a representative EGM signal illustrating timing intervals that may be used by an implantable medical device for measuring the recovery time and ARI associated with an extra systole.

FIG. 4C is a flow chart summarizing steps included in a calibration method for validating an extra systolic ARI measurement.

FIG. 4D is an alternative calibration method that may be used for validating an extra systolic ARI measurement and setting a recovery time sensing window.

FIG. 5 is a graph of a representative electrical restitution curve that may be constructed according to the method of FIG. 3.

FIG. 6 is a flow chart summarizing a method for automatically adjusting the operating extra systolic stimulation interval in response to changes in electrical restitution, which does not require reconstruction of the entire restitution curve.

FIG. 7 is a flow chart summarizing steps included in an alternative method for adjusting the operating extra systolic interval in response to transient changes in electrical restitution.

FIG. 8 is flow chart summarizing steps included in a general method for adjusting an extra systolic interval based on an index of electrical restitution.

FIG. 9 is an illustration of a representative EGM signal and corresponding time line depicting events occurring during extra systolic stimulation.

FIG. 10A is graph of activation-recovery intervals measured during a primary systole and an extra systole during extra systolic stimulation plotted versus the corresponding diastolic intervals.

FIG. 10B is a plot of activation-recovery intervals measured during a primary systole and an extra systole delivered at a relatively short diastolic interval.

FIG. 11 is a flow chart summarizing steps included in a method for automatically adjusting an operating extra systolic interval during extra systolic stimulation based on a measure of restitution kinetics derived from action potential duration related parameters measured during primary systoles and extra systoles.

FIG. 12 is a flow chart summarizing a method for optimizing the extra systolic interval during extra systolic stimulation based on both electrical restitution and mechanical enhancement of post-extra systolic beats.

FIG. 13 is a graph of the mechanical response to the extra systole plotted versus extra systolic interval and a corresponding graph of the electrical restitution curve.

FIG. 14 is a flow chart summarizing steps included in a method for controlling the extra systolic interval during extra systolic stimulation based on electrical and mechanical restitution curves.

FIG. 15 is a flow chart summarizing steps included in an alternative method for controlling the ESI based on electrical restitution and mechanical restitution.

FIG. 16 is a flow chart summarizing a method for controlling ESS according to previously-determined ESIs based on electrical restitution measurements made over a range of heart rates.

FIG. 17 is a flow chart summarizing steps included in a method for controlling ESS based on monitoring changes in the spatial dispersion of electrical restitution.

### DETAILED DESCRIPTION OF THE INVENTION

The present invention is directed toward providing an implantable system for delivering an electrical stimulation therapy to achieve post extra systolic potentiation (PESP) wherein the timing of the electrical stimulation therapy, referred to herein as “extra systolic stimulation” (ESS), is controlled based on measured electrical restitution properties of the myocardial tissue.

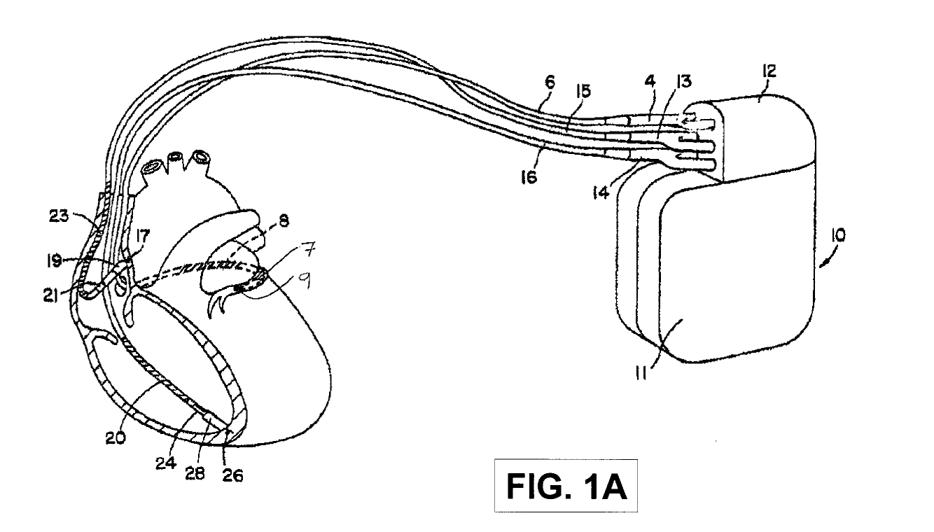

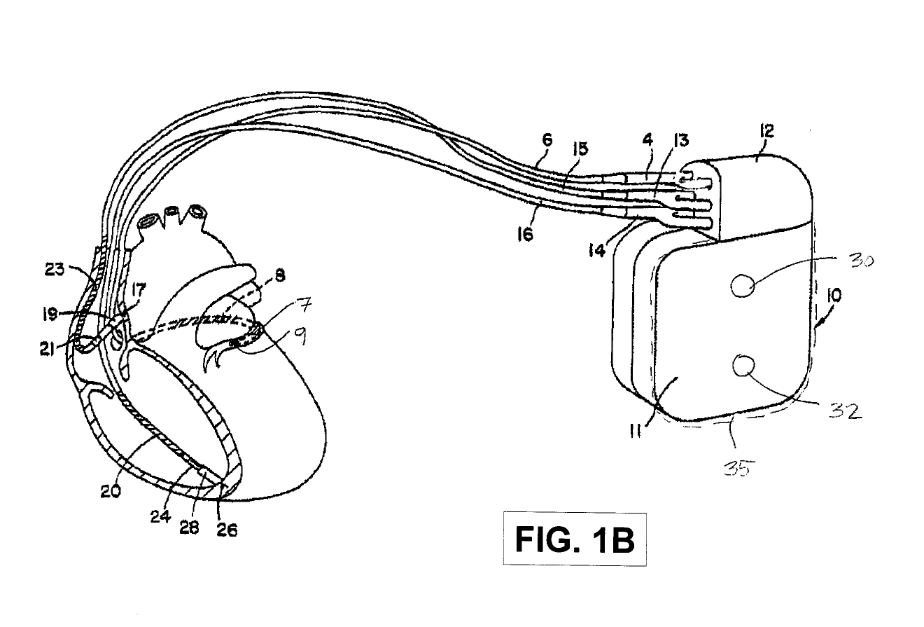

FIG. 1A is an illustration of an exemplary implantable medical device (IMD) in which the present invention may be implemented. IMD 10 is coupled to a patient's heart by three cardiac leads 6,15, and 16. IMD 10 is capable of receiving and processing cardiac electrical signals and delivering electrical stimulation pulses for ESS and may additionally be capable of cardiac pacing, cardioversion and defibrillation. IMD 10 includes a connector block 12 for receiving the proximal end of a right ventricular lead 16, a right atrial lead 15 and a coronary sinus lead 6, used for positioning electrodes for sensing and stimulating in three or four heart chambers.

In FIG. 1A, the right ventricular lead 16 is positioned such that its distal end is in the right ventricle for sensing right ventricular cardiac signals and delivering electrical stimulation therapies in the right ventricle which includes at least ESS and may include cardiac bradycardia pacing, cardiac resynchronization therapy, cardioversion and/or defibrillation. For these purposes, right ventricular lead 16 is equipped with a ring electrode 24, a tip electrode 26 optionally mounted retractably within an electrode head 28, and a coil electrode 20, each of which are connected to an insulated conductor within the body of lead 16. The proximal end of the insulated conductors are coupled to corresponding connectors carried by bifurcated connector 14 at the proximal end of lead 16 for providing electrical connection to IMD 10.

The right atrial lead 15 is positioned such that its distal end is in the vicinity of the right atrium and the superior vena cava. Lead 15 is equipped with a ring electrode 21, a tip electrode 17, optionally mounted retractably within electrode head 19, and a coil electrode 23 for providing sensing and electrical stimulation therapies in the right atrium, which may include atrial ESS and/or other cardiac pacing therapies, cardioversion and/or defibrillation therapies. In one application of PESP, ESS is delivered to the atria to improve the atrial contribution to ventricular filling. The extra systolic depolarization resulting from the atrial ESS stimulation pulse may be conducted to the ventricles for achieving PESP effects in both the atrial and ventricular chambers. The ring electrode 21, the tip electrode 17 and the coil electrode 23 are each connected to an insulated conductor with the body of the right atrial lead 15. Each insulated conductor is coupled at its proximal end to a connector carried by bifurcated connector 13.

The coronary sinus lead 6 is advanced within the vasculature of the left side of the heart via the coronary sinus and great cardiac vein. The coronary sinus lead 6 is shown in the embodiment of FIG. 1A as having a defibrillation coil electrode 8 that may be used in combination with either the coil electrode 20 or the coil electrode 23 for delivering electrical shocks for cardioversion and defibrillation therapies. Coronary sinus lead 6 is also equipped with a distal tip electrode 9 and ring electrode 7 for sensing functions and delivering ESS in the left ventricle of the heart as well as other cardiac pacing therapies. The coil electrode 8, tip electrode 9 and ring electrode 7 are each coupled to insulated conductors within the body of lead 6, which provides connection to the proximal bifurcated connector 4. In alternative embodiments, lead 6 may additionally include ring electrodes positioned for left atrial sensing and stimulation functions, which may include atrial ESS and/or other cardiac pacing therapies.

The electrodes 17 and 21, 24 and 26, and 7 and 9 may be used in sensing and stimulation as bipolar pairs, commonly referred to as a “tip-to-ring” configuration, or individually in a unipolar configuration with the device housing 11 serving as the indifferent electrode, commonly referred to as the “can” or “case” electrode. IMD 10 is preferably capable of delivering high-voltage cardioversion and defibrillation therapies. As such, device housing 11 may also serve as a subcutaneous defibrillation electrode in combination with one or more of the defibrillation coil electrodes 8, 20 or 23 for defibrillation of the atria or ventricles.

For the purposes of measuring electrical restitution in accordance with the present invention, an action potential duration related parameter is measured at differing extra systolic intervals from sensed cardiac electrical signals, preferably from a sensed EGM signal. An EGM signal may be sensed from a bipolar “tip-to-ring” sensing vector, a unipolar tip-to-can sensing vector, a unipolar tip-to-coil or ring-to-coil sensing vector, or a relatively more global coil-to-can sensing vector. A fiducial point on the QRS signal of the sensed EGM is used identify myocardial activation time, and a fiducial point on the T-wave is used to identify the myocardial recovery time. The interval between activation and recovery, referred to herein as the “activation-recovery interval” or ARI, is determined as an estimate of myocyte action potential duration. ARIs may be measured at the site which ESS will be delivered or at one or more alternative electrical restitution monitoring sites as will be described in greater detail below.

It is recognized that alternate lead systems may be substituted for the three lead system illustrated in FIG. 1A. For example, lead systems including one or more unipolar, bipolar and/or mulitpolar leads may be configured for sensing cardiac electrical signals from which an action potential duration related signal may be determined for measuring electrical restitution and for delivering ESS. It is contemplated that extra systolic stimuli may be delivered at one or more sites within the heart. Accordingly, lead systems may be adapted for sensing cardiac electrical signals for measuring restitution at multiple cardiac sites and for delivering extra systolic stimuli at the multiple sites, which may be located in one or more heart chambers. It is further contemplated that subcutanteous ECG electrodes could be included in the implantable system and that action potential duration related parameters determined from ECG signals may be used in measuring electrical restitution.

FIG. 1B is an illustration of an alternative IMD coupled to a set of leads implanted in a patient's heart. In FIG. 1B, IMD housing 11 is provided with an insulative coating 35, covering at least a portion of housing 11, with openings 30 and 32. The uninsulated openings 30 and 32 serve as subcutaneous electrodes for sensing global ECG signals, which may be used, in accordance with the present invention, for measuring electrical restitution. An implantable system having electrodes for subcutanteous measurement of an ECG is generally disclosed in commonly assigned U.S. Pat. No. 5,987,352 issued to Klein, incorporated herein by reference in its entirety. In alternative embodiments, multiple subcutaneous electrodes incorporated on the device housing 11 and/or positioned on subcutaneous leads extending from IMD 10 may be used to acquire multiple subcutaneous ECG sensing vectors for measurement of electrical restitution. Multi-electrode ECG sensing in an implantable monitor is described in U.S. Pat. No. 5,313,953 issued to Yomtov, et al., incorporated herein by reference in its entirety.

While a particular multi-chamber IMD and lead system is illustrated in FIGS. 1A and 1B, methodologies included in the present invention may be adapted for use with other single chamber, dual chamber, or multichamber IMDs that are capable of sensing and processing cardiac electrical signals and delivering electrical stimulation pulses at controlled time intervals relative to an intrinsic or paced heart rate. Such IMDs optionally include other electrical stimulation therapy delivery capabilities such as bradycardia pacing, cardiac resynchronization therapy, anti-tachycardia pacing, and preferably include arrhythmia detection and cardioversion, and/or defibrillation capabilities.

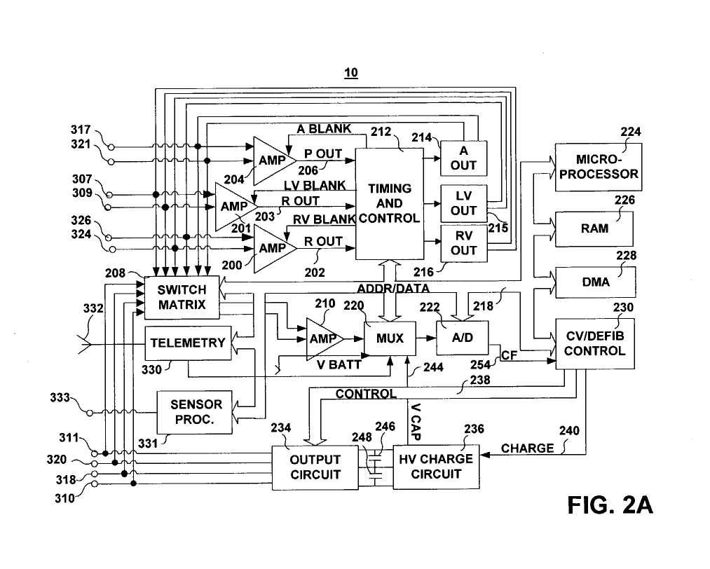

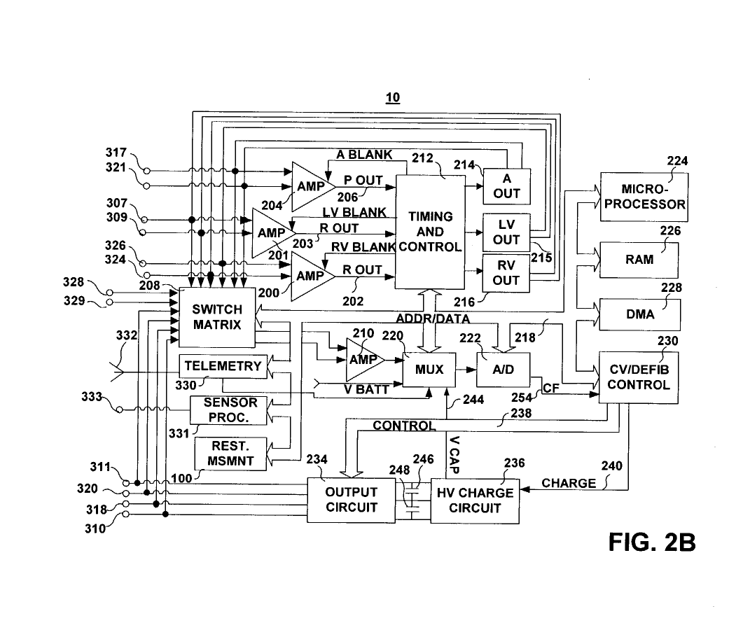

A functional schematic diagram of the IMD 10 is shown in FIG. 2A. This diagram should be taken as exemplary of the type of device in which the invention may be embodied and not as limiting. The disclosed embodiment shown in FIG. 2A is a microprocessor-controlled device, but the methods of the present invention may also be practiced in other types of devices such as those employing dedicated digital circuitry.

With regard to the electrode system illustrated in FIG. 1A, the IMD 10 is provided with a number of connection terminals for achieving electrical connection to the leads 6,15,16 and their respective electrodes. The connection terminal 311 provides electrical connection to the housing 11 for use as the indifferent electrode during unipolar stimulation or sensing. The connection terminals 320,310,318 provide electrical connection to coil electrodes 20,8,23 respectively. Each of these connection terminals 311, 320,310,318 are coupled to the high voltage output circuit 234 to facilitate the delivery of high energy shocking pulses to the heart using one or more of the coil electrodes 8,20,23 and optionally the housing 11. Connection terminals 311,320,310,318 are further connected to switch matrix 208 such that the housing 11 and respective coil electrodes 20,8,23 may be selected in desired configurations for various sensing and stimulation functions of IMD 10.

The connection terminals 317,321 provide electrical connection to the tip electrode 17 and the ring electrode 21 positioned in the right atrium. The connection terminals 317,321 are further coupled to an atrial sense amplifier 204 for sensing atrial signals such as P-waves. The connection terminals 326,324 provide electrical connection to the tip electrode 26 and the ring electrode 24 positioned in the right ventricle. The connection terminals 307,309 provide electrical connection to tip electrode 9 and ring electrode 7 positioned in the coronary sinus. The connection terminals 326,324 are further coupled to a right ventricular (RV) sense amplifier 200, and connection terminals 307,309 are further coupled to a left ventricular (LV) sense amplifier 201 for sensing right and left ventricular signals, respectively.

The atrial sense amplifier 204 and the RV and LV sense amplifiers 200,201 preferably take the form of automatic gain controlled amplifiers with adjustable sensing thresholds. The general operation of RV and LV sense amplifiers 200,201 and atrial sense amplifier 204 may correspond to that disclosed in U.S. Pat. No. 5,117,824, by Keimel, et al., incorporated herein by reference in its entirety. Generally, whenever a signal received by atrial sense amplifier 204 exceeds an atrial sensing threshold, a signal is generated on output signal line 206. P-waves are typically sensed based on a P-wave sensing threshold for use in detecting an atrial rate. Whenever a signal received by RV sense amplifier 200 or LV sense amplifier 201 that exceeds an RV or LV sensing threshold, respectively, a signal is generated on the corresponding output signal line 202 or 203. R-waves are typically sensed based on an R-wave sensing threshold for use in detecting a ventricular rate.

In one embodiment of the present invention, ventricular sense amplifiers 200,201 may include separate, dedicated sense amplifiers for sensing R-waves and T-waves, each using adjustable sensing thresholds, for the detection of myocardial activation and recovery times. Myocardial activation and recovery times are used in measuring an activation recovery interval (ARI) as an action potential duration related parameter for assessing electrical restitution as will be described in greater detail below. Myocardial activation times may be measured when a signal exceeding an activation time sensing threshold is received by an R-wave sense amplifier included in RV or LV sense amplifiers 200 or 201, causing a corresponding activation time sense signal to be generated on signal line 202 or 203, respectively. Likewise, recovery times may be measured when a signal exceeding a recovery time sensing threshold is received by a T-wave sense amplifier included in RV or LV sense amplifiers 200 or 201, causing a corresponding recovery time sense signal to be generated on signal line 202 or 203, respectively.

Switch matrix 208 is used to select which of the available electrodes are coupled to a wide band amplifier 210 for use in digital signal analysis. Selection of the electrodes is controlled by the microprocessor 224 via data/address bus 218. The selected electrode configuration may be varied as desired for the various sensing, pacing, cardioversion, defibrillation and ESS functions of the IMD 10. Signals from the electrodes selected for coupling to bandpass amplifier 210 are provided to multiplexer 220, and thereafter converted to multi-bit digital signals by A/D converter 222, for storage in random access memory 226 under control of direct memory access circuit 228. Microprocessor 224 may employ digital signal analysis techniques to characterize the digitized signals stored in random access memory 226 to recognize and classify the patient's heart rhythm employing any of the numerous signal processing methodologies known in the art. In accordance with the present invention, digital signal analysis of a selected EGM (or subcutaneous ECG signals if available) is performed by microprocessor 224 to derive ARIs for measuring electrical restitution.

The telemetry circuit 330 receives downlink telemetry from and sends uplink telemetry to an external programmer, as is conventional in implantable anti-arrhythmia devices, by means of an antenna 332. Data to be uplinked to the programmer and control signals for the telemetry circuit are provided by microprocessor 224 via address/data bus 218. Received telemetry is provided to microprocessor 224 via multiplexer 220. Numerous types of telemetry systems known for use in implantable devices may be used.

The remainder of the circuitry illustrated in FIG. 2A is an exemplary embodiment of circuitry dedicated to providing ESS, cardiac pacing, cardioversion and defibrillation therapies. The timing and control circuitry 212 includes programmable digital counters which control the basic time intervals associated with ESS, various single, dual or multi-chamber pacing modes, or anti-tachycardia pacing therapies delivered in the atria or ventricles. Timing and control circuitry 212 also determines the amplitude of the cardiac stimulation pulses under the control of microprocessor 224.

During pacing, escape interval counters within timing and control circuitry 212 are reset upon sensing of RV R-waves, LV R-waves or atrial P-waves as indicated by signals on lines 202,203,206, respectively. In accordance with the selected mode of pacing, pacing pulses are generated by atrial output circuit 214, right ventricular output circuit 216, and left ventricular output circuit 215. The escape interval counters are reset upon generation of pacing pulses, and thereby control the basic timing of cardiac pacing functions, which may include bradycardia pacing, cardiac resynchronization therapy, and anti-tachycardia pacing.

The durations of the escape intervals are determined by microprocessor 224 via data/address bus 218. The value of the count present in the escape interval counters when reset by sensed R-waves or P-waves can be used to measure R-R intervals and P-P intervals for detecting the occurrence of a variety of arrhythmias.

In accordance with the present invention, timing and control 212 further controls the delivery of extra systolic stimuli at selected extra systolic intervals (ESIs) following either sensed intrinsic systoles or pacing evoked systoles. The ESIs used in controlling the delivery of extra systolic stimuli by IMD 10 are preferably automatically adjusted by IMD 10 based on measurements of electrical restitution as will be described in greater detail below. The output circuits 214,215,216 are coupled to the desired stimulation electrodes for delivering cardiac pacing therapies and ESS via switch matrix 208.

The microprocessor 224 includes associated ROM in which stored programs controlling the operation of the microprocessor 224 reside. A portion of the memory 226 may be configured as a number of recirculating buffers capable of holding a series of measured R-R or P-P intervals for analysis by the microprocessor 224 for predicting or diagnosing an arrhythmia.

In response to the detection of tachycardia, anti-tachycardia pacing therapy can be delivered by loading a regimen from microcontroller 224 into the timing and control circuitry 212 according to the type of tachycardia detected. In the event that higher voltage cardioversion or defibrillation pulses are required, microprocessor 224 activates the cardioversion and defibrillation control circuitry 230 to initiate charging of the high voltage capacitors 246,248 via charging circuit 236 under the control of high voltage charging control line 240. The voltage on the high voltage capacitors is monitored via a voltage capacitor (VCAP) line 244, which is passed through the multiplexer 220. When the voltage reaches a predetermined value set by microprocessor 224, a logic signal is generated on the capacitor full (CF) line 254, terminating charging. The defibrillation or cardioversion pulse is delivered to the heart under the control of the timing and control circuitry 212 by an output circuit 234 via a control bus 238. The output circuit 234 determines the electrodes used for delivering the cardioversion or defibrillation pulse and the pulse wave shape.

In one embodiment, the implantable system may additionally include one or more physiological sensors for monitoring hemodynamic or myocardial contractile function or a metabolic status. The physiological sensor may reside within or on the heart, or endo- or extra-arterially for sensing a signal proportional to the hemodynamic function of the heart, myocardial contraction or heart wall motion, and/or a metabolic parameter. As such, IMD 10 is additionally equipped with sensor signal processing circuitry 331 coupled to a terminal 333 for receiving an analog sensor signal. A physiological sensor included in the implanted system may be, but is not limited to, a sensor of flow, pressure, heart sounds, wall motion, cardiac chamber volumes or metabolic parameters such as oxygen saturation or pH. Sensor signal data is transferred to microprocessor 224 via data/address bus 218 such that an index of cardiac hemodynamic or contractile performance or a metabolic status may be determined according to algorithms stored in RAM 226. Sensors and methods for determining a cardiac performance index as implemented in the previously-cited '098 patent to Bennett may also be used in conjunction with the present invention. As will be described in greater detail below, a mechanical or hemodynamic parameter of cardiac function or a metabolic parameter may be used in one embodiment of the present invention for controlling the ESI during ESS based on optimal mechanical enhancement of the post-extra systolic beats. In another embodiment of the present invention, control of the ESI includes measurement of the mechanical restitution during extra systoles.

Methods described herein for measuring electrical restitution may be implemented in software stored in RAM 226 executed by microprocessor 224. Alternatively, some or all operations for measuring electrical restitution may be implemented in dedicated circuitry. FIG. 2B is a functional schematic diagram of an alternative embodiment of the IMD 10, which includes dedicated circuitry for measuring electrical restitution. Electrical restitution measurement circuitry 100 is provided for receiving one or more EGM or subcutaneous ECG signals via switch matrix 208 and multiplexer 220 on address/data bus 218. In the embodiment of FIG. 2B and with regard to the electrode arrangement of FIG. 1B, connection terminals 328,329 are provided for connection to subcutaneous electrodes 30,32 incorporated in housing 11, for use in sensing ECG signals. EGM/ECG sensing vectors may be configured from any of the available electrodes via switch matrix 208. Restitution measurement circuitry 100 processes the one or more selected EGM/ECG signals for measuring an action potential duration related parameter and provides electrical restitution related data to microprocessor 224 for use in controlling ESS. Electrical restitution data may be stored in device memory 226 for later uplinking to an external device such that it is available for review by a physician for cardiac monitoring purposes.

As indicated above, an ARI may be measured as the action potential duration related parameter for measuring electrical restitution. As such, electrical restitution measurement circuitry 100 may include dedicated circuitry for detecting myocardial recovery times following extra systolic activation and measuring the intervening time interval. Recovery time detection circuitry may be provided as disclosed in co-pending non-provisional U.S. patent application Ser. No. 10/______ (Atty Dkt P-11214) to Burnes et al. filed on even date herewith, incorporated herein by reference in its entirety, which generally includes a T-wave feature detector and recovery time estimator.

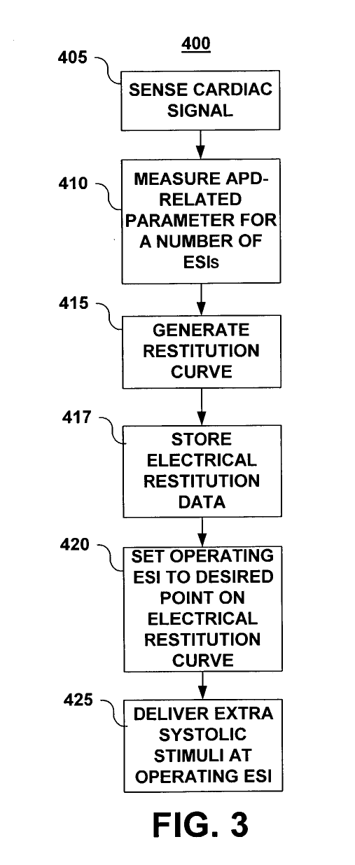

FIG. 3 is a flow chart providing an overview of methods included in one embodiment of the present invention for controlling ESS based on measurements of electrical restitution. At step 405, a signal of cardiac electrical activity is sensed for the purposes of deriving a measure of electrical restitution. The cardiac signal is preferably a cardiac EGM, which may be sensed at or near an ESS site or at other locations in the heart. If subcutaneous ECG electrodes are available, a subcutaneous ECG signal may be sensed at step 405 for measuring electrical restitution.

At step 410, an action potential duration (APD) related parameter is measured from the sensed signal for extra systoles delivered at a number of different extra systolic intervals (ESIs). As noted previously, electrical restitution can be described as the response of the action potential duration to changes in diastolic interval and reflects the recovery properties of the cardiac tissue to extra systoles. In a preferred embodiment of the present invention, a method for measuring electrical restitution takes advantage of the ability to reliably estimate myocyte action potential duration from a unipolar EGM signal. Thus, in a preferred embodiment, an action potential duration related parameter measured at step 410 is an activation-recovery interval (ARI) measured from a selected unipolar EGM, but may alternatively be measured from a bipolar, integrated bipolar or any other near-field or far-field EGM signal received from a sensing electrode configuration selected from any of the available electrodes included in an associated lead system. As will be further described below, the ARI measured between selected points on the QRS and T-wave of a unipolar EGM is well-correlated with local action potential duration. In alternative embodiments, ARIs are measured from a subcutaneous ECG signal. Action potential duration-related parameters may alternatively be measured according to methods known in the art, for example the methods taught generally in U.S. Pat. No. 6,152,882 issued to Prutchi, U.S. Pat. No. 6,522,904 issued to Mika et al., or U.S. Pat. No. 6,466,819 issued to Weiss.

In order to construct a restitution curve, ARIs for two or more ESIs are required. ARIs measured at step 410 may be measured during intrinsically occurring premature beats. The extra systolic intervals associated with the premature beats are measured as the interval between a sinus and premature beat. However, because the incidence of naturally-occurring premature beats may be random and infrequent, electrical restitution data collected in this way may require considerable time and may not represent a desired range of ESIs. Moreover, myocardial electrical restitution properties may vary over the time course required to collect electrical restitution data.

In a preferred embodiment, electrical restitution data is collected by proactively adjusting the ESI to a desired number of settings over a desired range and delivering ESS for a period of time or number of cardiac cycles at each ESI. Extra systolic stimuli may follow either or both sinus systoles or pacing-evoked systoles. Upon application of each ESI, a period of stabilization may be allowed prior to measuring the action potential duration related parameter to allow the myocardial response to the change in ESI to reach a steady state. Since restitution curve properties generally vary with heart rate, a family of restitution curves may be acquired for different sinus and/or paced heart rates.

At step 415, an electrical restitution curve is generated by plotting the measured action potential duration related parameter versus the intrinsic or applied ESIs. At step 417, electrical restitution data is preferably stored with a time and date label for diagnostic purposes. Stored electrical restitution data may include the action potential duration related data and ESIs used to generate a restitution curve, and/or selected characteristic points, measured slopes and/or other characteristics of the restitution curve. Additional patient-related or other physiological data may be stored with the electrical restitution data, such as the patient's heart rate or pacing rate, activity level, blood pressure, etc. Stored data is made available for display on an external device and review by a clinician upon receiving an interrogation command from an external programmer.

An operating ESI setting is then selected based on the electrical restitution curve at step 420. The ESI setting is selected to correspond to a desired operating point on the restitution curve. In a preferred embodiment, the operating ESI is set as the shortest ESI on the plateau phase of the restitution curve. Shorter ESIs, occurring on the steep phase of the restitution curve are preferably avoided since heightened action potential duration shortening associated with this portion of the curve can result in greater heterogeneity of refractoriness creating a substrate for re-entrant arrhythmias. Other operating points that may be selected include, but are not limited to, the transition point between the steep slope and plateau phases of the restitution curve or the peak or trough of a biphasic “hump” on the restitution curve.

At step 425, ESS is delivered at the operating ESI and according to other programmed ESS operating parameters, which may include the extra systolic stimulus pulse width, pulse amplitude, the ratio of extra systolic stimuli to the normal sinus or paced heart rate, ESS “on” and “off” periods, etc. Changes in disease state, medical therapy, or a number of other physiological influences may alter the myocardial electrical restitution properties over time. Therefore, method 400 may be repeated on a periodic basis, or at any time upon receiving a user command from an external programmer, to reconstruct the electrical restitution curve and adjust the ESI setting so that it remains at the desired operating point on the restitution curve.

It is recognized that method 400 of FIG. 3 may be applied to multiple cardiac sites, within one or more cardiac chambers for measuring and storing related electrical restitution data for diagnostic purposes and/or for setting ESIs for applying ESS at multiple cardiac sites. By measuring and storing electrical restitution data for two or more cardiac sites, spatial dispersion of electrical restitution can be measured to assess the propensity of the heart for arrhythmias.

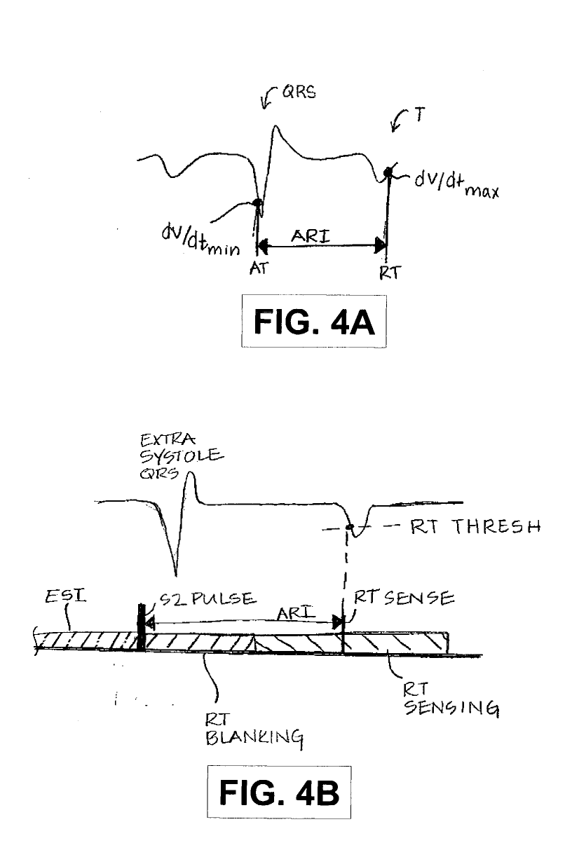

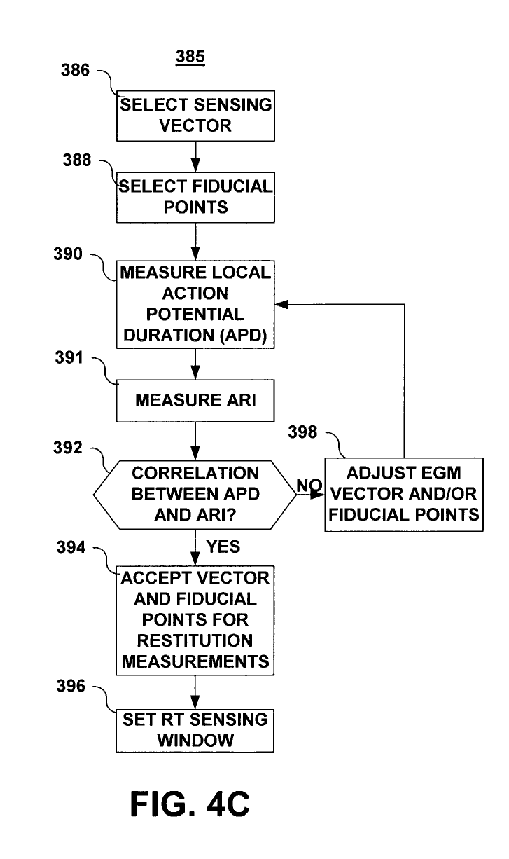

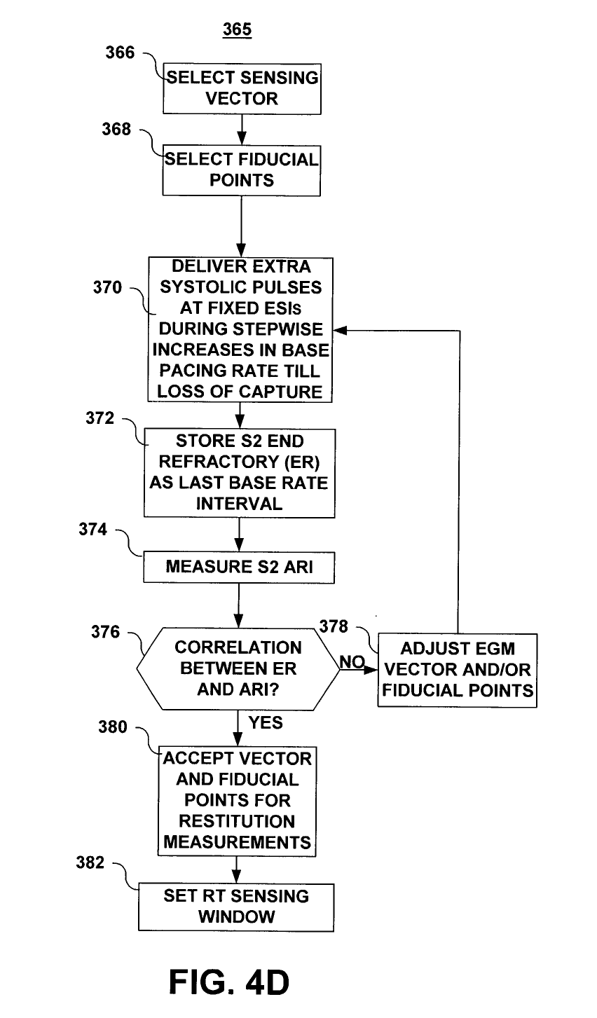

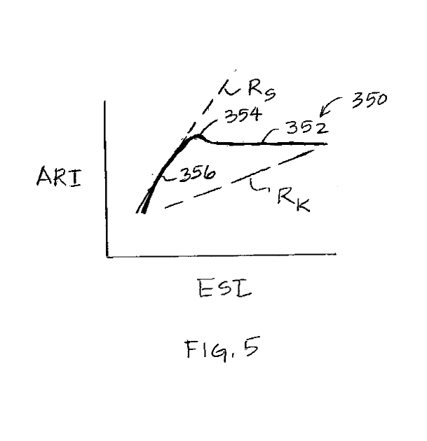

FIG. 4A depicts a representative unipolar EGM signal illustrating one method for measuring the activation recovery interval which may be employed by method 400 of FIG. 3 in collecting electrical restitution data. Details regarding methods for measuring ARIs appropriate for use in the present invention are also described in co-pending non-provisional U.S. patent application Ser. No. 10/______ (Atty Dkt P-11215.00) to Burnes, incorporated by reference herein in its entirety, and as described in the previously-incorporated non-provisional application to Burnes et al. (Atty Dkt P-11214.00). Briefly, and as shown in FIG. 4, a fiducial point on a QRS signal is selected for measuring myocardial activation time (AT). This point is preferably the maximum negative derivative of the QRS signal, dV/dtmin, on a unipolar EGM, but may alternatively be a maximum or minimum peak, a maximum positive derivative, a threshold crossing, or other fiducial point identifiable on a sensed EGM or subcutaneous ECG signal. A fiducial point on the T-wave is selected for measuring myocardial recovery time (RT). This point is preferably the maximum positive derivative of the T-wave, dV/dtmax, on a unipolar EGM, but may alternatively be a maximum or minimum peak, a maximum negative derivative, a threshold crossing, or other fiducial point identifiable on a sensed unipolar or bipolar EGM signal or subcutaneous ECG.

The difference between the AT and RT is determined as the ARI. ARI measured as the interval on a unipolar EGM between the maximum negative derivative of the QRS signal and the maximum positive derivative of the T-wave is closely correlated to the duration of the local monophasic action potential. With regard to the present invention, the extra systolic ARI may be measured as the interval between the extra systolic stimulation pulse, rather than a detected activation time on the extra systolic QRS, and a detected extra systolic recovery time.

Additional details for measuring an ARI that may be usefully practiced in measuring the extra systolic ARI in the present invention are described in the two co-pending non-provisional U.S. patent applications (Atty Dkts P-11214.00 and P-11215.00). For example, the detection of the recovery time following an extra systole may include the use of a recovery time detection window and/or a recovery time blanking window. FIG. 4B is a timing diagram shown in temporal relation to a representative EGM signal illustrating timing intervals that may be used by an implantable medical device for measuring activation time, recovery time and ARI associated with an extra systole. The activation time in this embodiment is determined as the time of delivering an extra systolic stimulation pulse (S2 pulse) at the end of an ESI. A fiducial point on the evoked QRS signal following the S2 pulse may alternatively be detected as the activation time. For example, the activation time may alternatively be detected as the point at which the R-wave crosses an activation time sensing threshold, or at which an R-wave peak, valley, maximum or minimum slope, or other identifiable fiducial point occurs, as described previously.

In this embodiment, a method for detecting recovery time employs the use of timing intervals set relative to the detected activation time for narrowing the search for recovery time. Immediately following the S2 pulse (or a detected activation time), a recovery time blanking period (RT BLANKING) may be applied for a time interval following the S2 pulse during which recovery is not expected to occur because it is too early after activation.

After the blanking period has expired, recovery time sensing is enabled during a recovery time sensing window (RT SENSING). In one embodiment, a recovery time sensing window may be positioned in time such that it is approximately centered over the expected time occurrence of the extra systolic T-wave. In an alternative embodiment, the recovery time sensing may be enabled upon expiration of the blanking period and remain enabled until an extra systolic recovery time is detected according to a fiducial point on the extra systolic T-wave or until the next primary systole is detected. In the embodiment shown in FIG. 4B, the fiducial point for detecting recovery time is the point that the extra systolic T-wave crosses a recovery time sensing threshold (RT THRESH). Alternative fiducial points as described above may be searched for during a recovery time sensing window for detecting recovery time. If another activation time (associated with a primary systolic event) is detected prior to detecting an extra systolic recovery time, the recovery time blanking and recovery time sensing window are reset to begin looking for the next recovery time following the next ESS pulse (S2). The extra systolic ARI for the current S2 pulse is not measured because the extra systolic recovery time has gone undetected.

If the recovery time is to be detected using digital signal analysis of the sensed EGM signal, the recovery time sensing window may define the beginning and end of an EGM signal segment to be digitized for searching for the fiducial point on the T-wave identified as recovery time. If the recovery time is detected using a dedicated sense amplifier having an adjustable recovery time sensing threshold, the recovery time blanking window and the recovery time sensing window define intervals of time during which the dedicated sense amplifier is blanked or enabled, respectively. The extra systolic ARI is measured as the interval between the ESS pulse (or a detected activation time) and the detected recovery time (RT).

FIG. 4C is a flow chart summarizing steps included in a calibration method for validating an ARI measurement. The calibration method 385 may be performed under clinical supervision for ensuring that the selected sensing vectors and fiducial points used for detecting extra systolic recovery time provide an extra systolic ARI measurement that correlates with the action potential duration at or near the ESS site. At step 386, an initial sensing vector for detecting extra systolic recovery times (and optionally activation time) is selected corresponding to a desired monitoring site, which may be a near-field or far-field EGM or subcutaneous ECG signal. At step 388, fiducial points are selected for detecting extra systolic recovery time on the sensed signal received from the selected sensing vector. If a detected activation time is to be used for measuring the extra systolic ARI rather than the time of the ESS pulse, a fiducial point for detecting the extra systolic activation time may also be selected at step 388.

At step 390, an action potential duration (APD) at or near an ESS site is measured for an extra systole delivered at a reference ESI using a reference electrode system. Any known electrophysiological method for making reliable, acute measurements of local action potential duration may be used. At step 391, an ARI is measured using the selected sensing vector and fiducial point for recovery time, using the methods described above. Repeated measurements of the APD and the extra systolic ARI may be made at steps 390 and 391 at the same or different heart rates to acquire a series of measurements to establish the correlation between the APD and the ARI measurements. APD and ARI measurements may be performed simultaneously on the same cardiac cycle or sequentially, under stable physiological conditions.

At decision step 392, the APD(s) measured at step 390 are compared to the ARI(s) measured at step 391 to determine if the ARI measured using the selected sensing vector and fiducial point for recovery time detection is approximately equal to or correlates with the APD measurement. If the APD and ARI measurements differ by more than an acceptable amount, for example by more than a predetermined percentage, the sensing vector and/or the fiducial points selected for measuring recovery time (and optionally activation time) are adjusted at step 398. The APD measurement(s) are repeated at step 390, and ARI(s) are measured at step 391 using the adjusted measurement parameters.

Once a satisfactory correlation between the APD and ARI measurements is obtained, as determined at decision step 392, the currently selected sensing vector and fiducial points for detecting activation time and recovery time are accepted, at step 394, as the operating measurement parameters for measuring the extra systolic ARI for use in measuring electrical restitution.

At step 396, the measurement of the APD may be used for setting a recovery time sensing window that is used in searching for the fiducial point on the T-wave for recovery time detection. The recovery time sensing window may be set such that it is centered approximately on the end point of the local action potential duration. Alternatively, a recovery time sensing window is set to at least begin earlier than the end of the local action potential duration.

Calibration method 385 may be repeated for multiple sites for evaluating electrical restitution at multiple monitoring or ESS sites. Steps included in method 385 may be performed only for the purposes of verifying the selection of a sensing vector and/or fiducial points for detecting recovery time (and optionally activation time) without setting a recovery time sensing window. Alternatively, steps included in method 385 may be performed for setting a recovery time sensing window without adjusting sensing vector or fiducial point selections.

FIG. 4D is an alternative calibration method that may be used for validating an extra systolic ARI measurement and setting a recovery time sensing window. At steps 366 and 368, respectively, an initial sensing vector and fiducial points for detecting extra systolic recovery time (and optionally activation time) for a given monitoring or ESS site are selected. At step 370, extra systolic stimulation pulses are delivered at a selected site. Extra systolic stimulation pulses are inserted at a fixed ESI in a train of primary pacing pulses. Primary pacing pulses are delivered at a step-wise increasing base rate such that the diastolic interval following the extra systole is progressively shortened. Alternatively, only the first post extra systolic pacing pulse is delivered at a decreasing base rate (or shortened escape interval). Because delivering pacing pulses at short intervals following the extra systole can induce an arrhythmia, method 365 is preferably performed under clinical supervision.

The primary pacing escape interval is decreased until capture is lost on the first post-extra systolic primary pacing pulse. When the shortened escape interval causes the first post-extra systolic pacing pulse to be delivered during the extra systolic refractory period, capture is lost. The escape interval at which capture is lost is determined as an approximate measure of the end of the local refractory period following the extra systolic depolarization. At step 372, the last escape interval, which resulted in loss of capture, is stored as the extra systolic end refractory time. At step 374, the extra systolic ARI is measured using the selected sensing vector and fiducial point for recovery time detection (and optionally activation time detection). The extra systolic end refractory time is compared to the measured ARI at decision step 376 to verify that the measured ARI is within an acceptable range of the approximate end refractory time (ER).

If the measured ARI and stored end refractory time are not approximately equal, the sensing vector and/or fiducial points for detecting recovery time (and/or activation time) may be adjusted at step 378. Steps 370 through 376 are repeated until an acceptable correlation between the measured end refractory time and ARI is obtained, as determined at decision step 376. Alternatively, steps 370 and 372 are performed once and, as long as stable physiological conditions remain, method 365 may loop back to step 374 from step 378 to only repeat ARI measurements at adjusted measurement parameters until satisfactory agreement is reached between an ARI measurement and the previously measured end refractory time. Once a satisfactory correlation between the measured end refractory time and ARI is obtained, the currently selected sensing vector and fiducial points for detecting recovery time (and optionally activation time) are accepted at step 380 as the operating measurement parameters for measuring the extra systolic ARI for the given ESS or monitoring site, for used in measuring electrical restitution.

At step 382, a recovery time sensing window may be set based on the measurement of the extra systolic end refractory time. In one embodiment, the recovery time sensing window is set such that it is centered approximately on the end refractory time. Alternatively, a recovery time sensing window is set to at least begin earlier than the end of the extra systolic refractory time.

Method 365 may be repeated for each site to be included in multi-site ESS or restitution monitoring. Steps included in method 365 may be performed only for the purposes of selecting the sensing vector and/or fiducial points for detecting recovery time without setting a recovery time sensing window. Alternatively, steps included in method 365 may be performed for setting a recovery time sensing window without adjusting sensing vector or fiducial point selections.

FIG. 5 is a graph of a representative electrical restitution curve that may be constructed according to method 400 of FIG. 3. Restitution curves are traditionally constructed by plotting the measured APD from the extra-systolic beat against the diastolic interval associated with the extra-systole. The diastolic interval (DI) is defined as the extra-systolic interval minus the APD of the primary systolic beat. If the APD of the systolic beat is assumed constant, ESI could be substituted for DI. In FIG. 5, the ESI is used for the construction of the restitution curves, but DI could be used as well. Measured extra systolic activation recovery intervals are plotted against applied extra systolic intervals producing restitution curve 350. Restitution curve 350 is typically characterized by a relatively steep phase 356 associated with heightened shortening of the ARI with relatively short ESIs and a plateau phase 352 associated with a maximum ARI at longer ESIs. The peak of the steep phase may be followed by a decrease in ARI before reaching the plateau phase 352 forming a biphasic “hump” 354.

By obtaining two or more points along the restitution curve, a measure of restitution can be derived. Measures of restitution include the slope of the steep phase of a restitution curve, referred to as “Rₛ”, and the slope of the overall restitution curve, referred to as “R_{K}”. In accordance with the present invention, R_{K} may be calculated according to the following equation:

R_{K}=(ARIₘₐₓ−ARIₘᵢₙ)/(ESIₘₐₓ−ESIₘᵢₙ)

wherein ARIₘₐₓ and ARIₘᵢₙ are the maximum and minimum extra systolic ARIs measured at the maximum applied ESI, ESIₘₐₓ, and the minimum applied ESI, ESIₘᵢₙ, respectively.

As noted above, electrical restitution properties may vary over time. Because the maximum PESP effect is gained when the extra systolic stimulus is delivered just after recovery from the primary systole, it is desirable to maintain the operating ESI near the left-hand side of the plateau 352 on the restitution curve or at a selected point on or relative to the biphasic “hump” 354 of the curve.

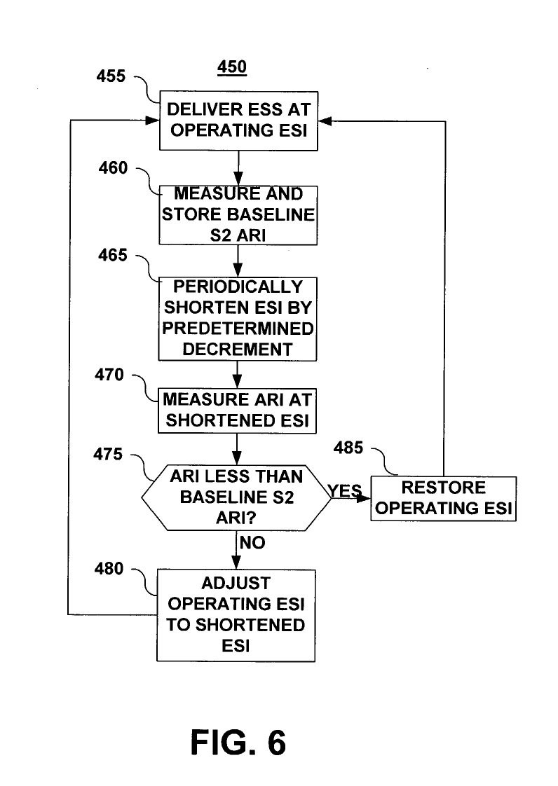

FIG. 6 is a flow chart summarizing a method for automatically adjusting the operating ESI in response to changes in electrical restitution, which does not require reconstruction of the entire restitution curve. Method 450 begins at step 455 by delivering ESS at the operating ESI, which has been determined previously based on the electrical restitution curve according to method 400 of FIG. 3. At step 460, the ARI of the extra systole, S2, delivered at the operating ESI is stored as the baseline S2 ARI. The S2 ARI may be stored from the restitution curve data obtained during the operations of method 400 of FIG. 3, or the S2 ARI may be re-measured after reaching a steady state during ESS at the operating ESI. The baseline S2 ARI may be measured from a single cardiac cycle or determined as an average of the S2 ARI measured during several cardiac cycles after reaching steady state.

According to method 450, during ESS operations, the ESI is periodically shortened from the operating setting by a predetermined decrement, for example on the order of 10 ms. The ARI of the extra systole is measured at the shortened ESI at step 470 after an optional period of stabilization, which may be on the order of a few seconds to a few minutes.

At step 475, the measured S2 ARI at the shortened ESI is compared to the baseline S2 ARI. If the S2 ARI at the shortened ESI is substantially less than the baseline S2 ARI, the operating ESI is restored at step 485. Method 450 returns to step 455 to deliver ESS at the original operating ESI. If, however, the measured ARI is not substantially less than the baseline S2 ARI, the operating ESI is adjusted to the shortened ESI at step 480. Method 450 returns to step 455 to deliver ESS at the new operating ESI equal to the shortened ESI.

By occasionally shortening the ESI, method 450 is probing to the left on the electrical restitution curve to determine if the shortened ESI is still on the desired plateau phase of the restitution curve. If the S2 ARI decreases substantially, then the shortened ESI is occurring on the steep phase of the restitution curve, which is generally undesirable. In this way, method 450 automatically maintains an operating ESI setting at the shortest available setting on the plateau phase of the restitution curve.

Baseline S2 ARIs stored for each operating ESI setting may be reviewed by a clinician for diagnostic purposes. Stored ARI data may be uplinked from the implanted medical device to the external device for display and review upon receipt of an interrogation command from the external programmer. Changes in the S2 ARI reflect alterations in the electrical restitution properties of the heart, which may be the result of a change in disease state or a response to a medical therapy, a cardiac stimulation therapy, or other delivered therapies.

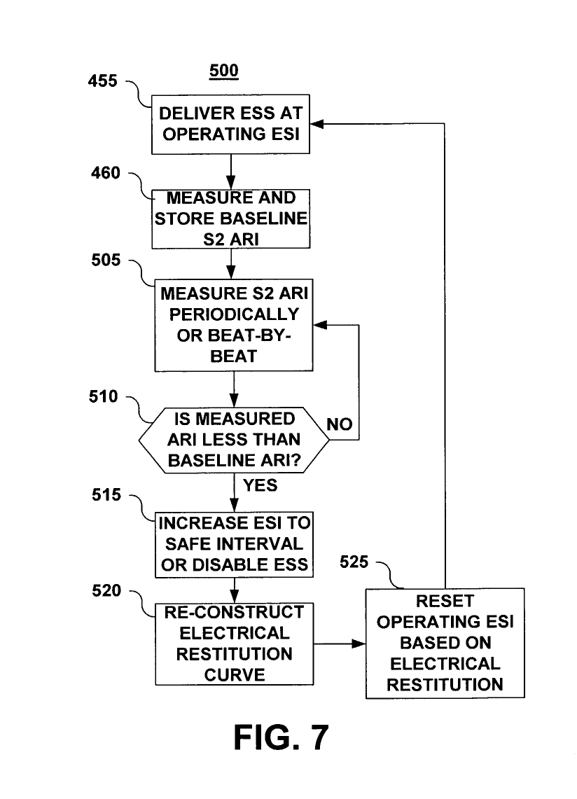

FIG. 7 is a flow chart summarizing steps included in an alternative method for adjusting the operating ESI in response to transient changes in electrical restitution. Steps 455 and 460 correspond to the identically labeled steps of method 450 described above. Briefly, ESS is delivered at a previously-determined operating ESI at step 455, and a baseline ARI interval associated with the extra systole at the operating ESI is stored at step 460.

According to method 500, changes in electrical restitution are monitored by monitoring for changes in the S2 ARI at the operating ESI. As such, the ARI of the extra systole is measured at step 505 on a beat-by-beat or less frequent basis. In an alternative embodiment, a running average of the ARI during the extra systole is determined by averaging a predetermined number of consecutively measured ARIs at step 505. The measured S2 ARI is compared to the baseline S2 ARI at decision step 510. If the measured S2 ARI (or S2 ARI running average) is not significantly less than the baseline S2 ARI, method 500 continues delivering ESS at the operating ESI and monitoring for abrupt changes in the S2 ARI by returning to step 505.

If the measured or running average S2 ARI is significantly less than the baseline S2 ARI, as determined at decision step 510, a change in the electrical restitution properties may have occurred, shifting the operating ESI onto the steep phase of the restitution curve. Therefore, at step 515, the ESI is temporarily increased to a known safe interval. A known safe interval may be, for example, a predetermined fixed interval, a multiple of the operating ESI, or set based on the current intrinsic heart rate or pacing rate. Alternatively, ESS is temporarily disabled at step 515 while an electrical restitution curve is reconstructed at step 520 according to method 400 described previously in conjunction with FIG. 3. Data used for constructing the restitution curve or restitution data derived from the curve may be stored at step 520 for diagnostic purposes as described previously. The operating ESI is adjusted to the desired point on the new restitution curve at step 525. Method 500 then returns to step 455 to continue delivering ESS at the new operating ESI. Thus method 500 allows sudden changes in restitution to be promptly detected and responded to by adjusting the ESI and/or temporarily suspending ESS.

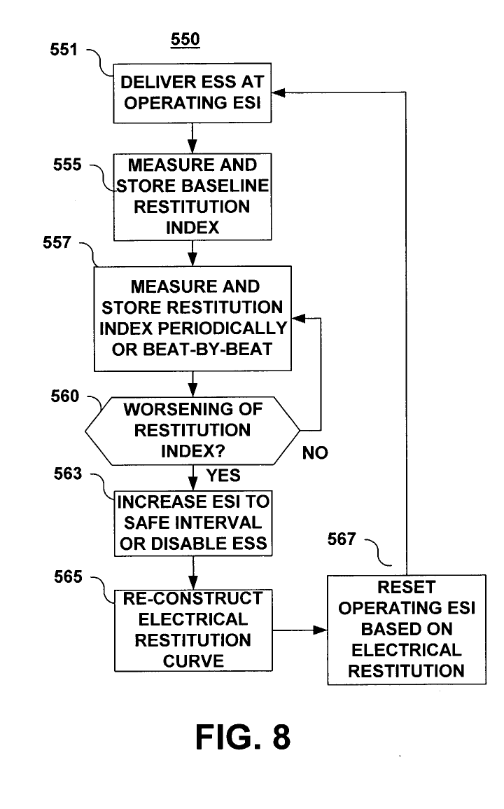

FIG. 8 is flow chart summarizing steps included in a general method for adjusting an ESI based on an index of electrical restitution. According to method 550, an index of electrical restitution is determined from one or more points on the restitution curve and is used to monitor for changes in restitution. Indices of restitution may include, but are not limited to, the slopes R_{S} and R_{K} shown in FIG. 5.

Method 550 begins at step 551 by delivering ESS at a previously-determined operating ESI. At step 555, a restitution index is stored based on the restitution curve measured for setting the operating ESI. During ESS delivery, the restitution index is re-determined on a periodic or beat-by-beat basis at step 557. A restitution curve may be updated continuously based on ARIs measured for each extra systole. Alternatively, an iterative procedure may be performed periodically for reconstructing the restitution curve by measuring the ARI of the extra systole at varying ESIs. Depending on the restitution index being monitored, one or more characteristic points on the restitution curve may be determined in order to determine the restitution index.

At decision step 560, method 550 determines if the measured restitution index indicates a worsening of restitution. A worsening of restitution may be measured by comparing the restitution index to the stored baseline index, a previously measured index, or by comparing the measured index to a predetermined threshold level. Depending on the method of determining the index, a worsening of restitution may be associated with an increase or a decrease of the index. For example, if a slope R_{S} or R_{K}, is calculated as an index of electrical restitution, an increase in slope generally indicates a worsening in electrical restitution toward a state that may be more arrhythmogenic.

If the restitution index remains relatively unchanged compared to the baseline index, ESS at the operating ESI continues at step 557 with periodic or beat-by-beat monitoring of the restitution index. If a significant change in the restitution index is measured at decision step 560 indicating a worsening of restitution, the ESI is increased to a relatively long, safe interval or ESS is temporarily suspended at step 563. At step 565, the electrical restitution curve is reconstructed, and the operating ESI is adjusted to the desired operating point on the new restitution curve at step 567. Restitution data, including measured restitution indices, may be stored for diagnostic purposes as described previously. Method 550 then returns to step 551 to deliver ESS at the new operating ESI.

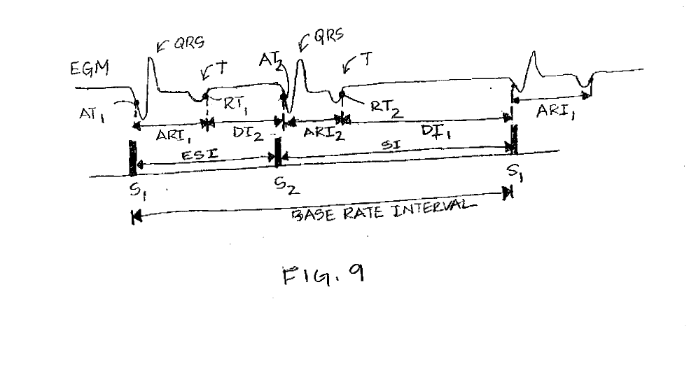

FIG. 9 is an illustration of a representative EGM signal and corresponding time line depicting events occurring during ESS. A primary systolic event, S1, which may be an intrinsic heart beat or a pacing pulse, is shown on the time line followed by an extra systolic stimulation pulse, S2, which is separated in time from the S1 event by an ESI. The QRS wave and T-wave on the EGM signal associated with the S1 event are indicated. The activation-recovery interval (ARI₁) associated with the S1 event is the interval between the activation time, AT₁, and the recovery time, RT₁, detected on the QRS and T-waves, respectively, according to the methods described previously for measuring ARIs. The ESI is the sum of the ARI₁ plus a short diastolic interval, DI₂, occurring between S1 recovery and S2 activation. The activation-recovery interval (ARI₂) associated with the extra systole, S2, is the interval between the activation time, AT₂, and the recovery time, RT₂, detected on the QRS and T-waves of the EGM signal following S2.

In FIG. 9, a second primary S1 event is shown following the extra systole S2. The two S1 events occur at a base rate interval corresponding to a base pacing rate or the intrinsic heart rate. The difference between the base rate interval and the ESI associated with the intervening extra systole is equal to a systolic interval (SI) between the extra systole, S2, and the second S1 event. The SI is the equal to the sum of ARI₂ and the subsequent diastolic interval, DI₁. As the heart rate or pacing rate change, the base rate interval will change resulting in changes in the SI. Thus, it is seen by the illustration of FIG. 9 that two ARIs, an ARI₁ and an ARI₂, may be measured during ESS. These two ARIs, one measured during the extra systole S2 occurring at a short diastolic interval, DI₂, and the other measured during the primary systole S1 occurring at a longer diastolic interval, DI₁, provide two points on a restitution curve. These two points may advantageously be used in monitoring for transient changes in electrical restitution and appropriately adjusting the operating ESI based on such transient changes.

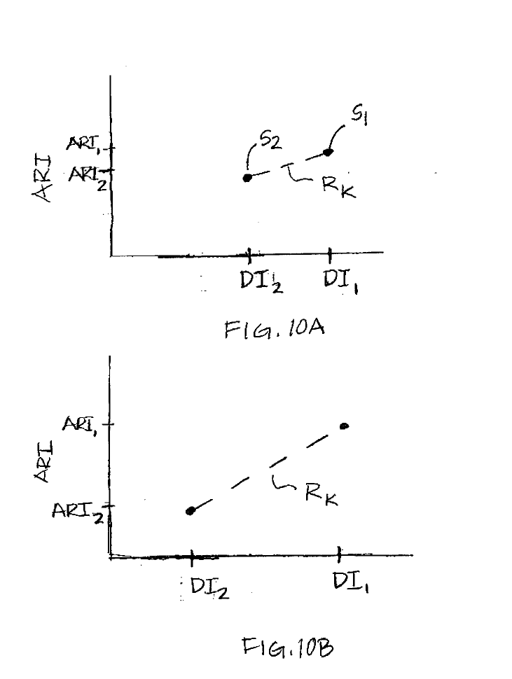

FIG. 10A is graph of ARIs measured during a primary systole and an extra systole during ESS plotted versus the corresponding diastolic intervals. A point on the restitution curve associated with the extra systole, S2, is plotted as the ARI₂ measured from an EGM signal during the extra systole. A second point on the restitution curve associated with the primary systole, S1, is plotted as the ARI₁ measured during the primary systole from the same EGM signal. A measure of restitution kinetics can be measured as the slope, Rₖ, of the line connecting these two points:

(2) Rₖ=(ARI₁−ARI₂)/(DI₁−DI₂).

FIG. 10B is a plot of ARIs measured during a primary systole and an extra systole delivered at a relatively short ESI. The resulting slope R_{K}, is much steeper due to the exaggerated shortening of the extra systolic ARI at the short diastolic interval, DI₂. As the slope Rₖ increases towards 1, the risk of re-entrant arrhythmias increases. In one embodiment of the present invention, the operating ESI is adjusted in order to maintain the slope R_{K} near 0 or alternatively less than some predefined safe limit. Thus, the relationship between the ARIs measured during primary and extra systoles may be monitored on a beat-by-beat or less frequent basis for detecting changes in the myocardial electrical restitution properties. As myocardial electrical restitution properties vary, action potential durations associated with the primary systole and the extra systole may both change. Therefore, the ESI may be adjusted based on a measure of restitution determined from the relation between the primary and extra systolic ARIs.

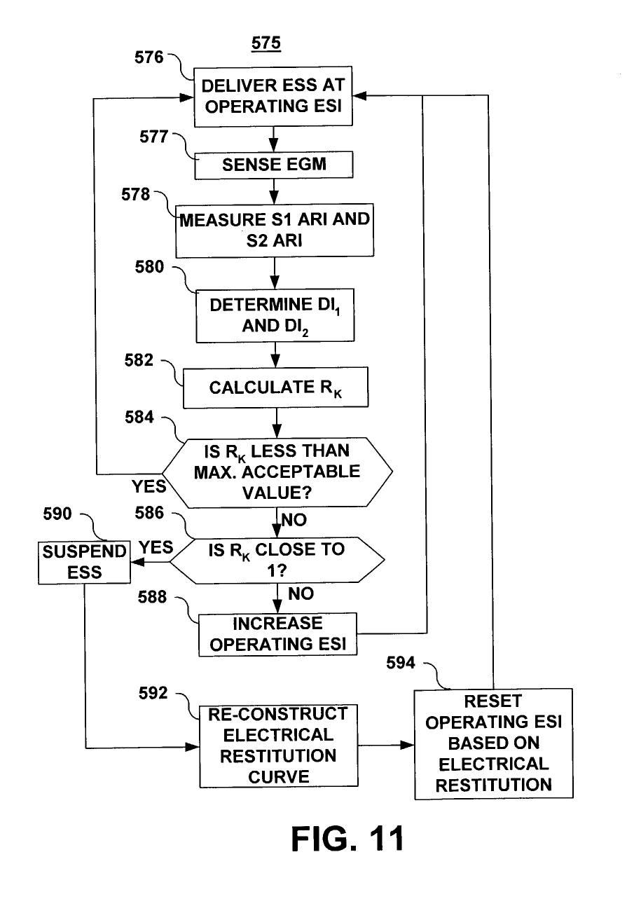

FIG. 11 is a flow chart summarizing steps included in a method for automatically adjusting an operating ESI during ESS based on a measure of restitution kinetics derived from action potential duration related parameters measured during primary systoles and extra systoles. Method 575 begins at step 576 with the delivery of ESS at a predetermined operating ESI. An EGM signal selected for monitoring electrical restitution is sensed at step 577. At step 578, the primary systole (S1) ARI and the extra systole (S2) ARI are measured from the sensed EGM signal. Both the S1 ARI and the S2 ARI are determined from the same EGM signal in order to evaluate and detect changes in restitution.

At step 580, the primary diastolic interval, DI₁, and the extra systolic diastolic interval, DI₂, are determined. DI₁ is determined as the base rate interval (intrinsic or paced), occurring between two consecutive primary systolic events, less the ESI and the extra systolic ARI (ARI₂). DI₂ is determined as the ESI less the primary systolic ARI (ARI₁).

At step 582, the slope R_{K} of the restitution curve is calculated according to equation (2) above using the measured S1 and S2 ARIs and the calculated diastolic intervals. The calculated slope R_{K} is compared to a predetermined maximum acceptable level at decision step 584. If R_{K} is less than the maximum acceptable level, method 575 returns to step 576 and continues to deliver ESS at the current operating ESI. The operating ESI is located at an acceptable point on the restitution curve based on the calculated slope between the S1 and S2 points.

If however, the slope R_{K} is greater than an acceptable level, as determined at decision step 586, method 575 determines if the slope is close to 1, or alternatively greater than some predetermined critical value at decision step 586. If slope R_{K} is close to 1 or greater than some critical value, then ESS is temporarily suspended at step 590. A slope R_{K} close to 1 indicates that the operating ESI is located at an unacceptable point on the steep portion of the restitution curve. ESS is suspended due to the greater arrhythmia risk. The restitution curve may be reconstructed at step 592 to allow the operating ESI to be reset at a desired point on the restitution curve at step 594. Restitution data, including calculated R_{K} values, may be stored at step 592 as described previously for diagnostic purposes.

If the slope R_{K} calculated at step 582 is greater than the maximum acceptable value (decision step 584) but not greater than a critical value (decision step 586), the operating ESI may be located near the transition point of the restitution curve. Therefore, the operating ESI is increased at step 588, preferably by a predetermined increment, such as an increment on the order of 10 ms. ESS stimulation is continued at step 576 at the new operating ESI and method 575 repeats. Additional increases in ESI may be made as needed according to the calculated slope R_{K}. Thus method 575 allows adjustments of the ESI to be made based on a determination of restitution slope R_{K}, which may be determined as frequently as every cardiac cycle that includes an extra systolic beat.

Measuring electrical restitution and monitoring transient changes in restitution according to the present invention provide a basis for safely delivering ESS by automatically adjusting the ESI in order to avoid increased arrhythmia risk. However, the goal of ESS therapy is to safely achieve mechanical enhancement of cardiac function on post-extra systolic beats. As such, it is recognized that methods described herein for controlling the adjustment of the ESI based on electrical restitution may additionally include methods for adjusting the ESI for achieving maximum enhancement of myocardial contraction on post-extra systolic beats. Generally, the minimum safe ESI, or a desired operation range, is determined based on electrical restitution properties. The ESI setting is then optimized for achieving maximum PESP effects based on monitoring hemodynamic or myocardial contractile function, within the bounds set forth based on electrical restitution. Thus, an optimal ESI setting can be determined based on both electrical restitution measurements and measurements of mechanical heart function on post-extra systolic beats.

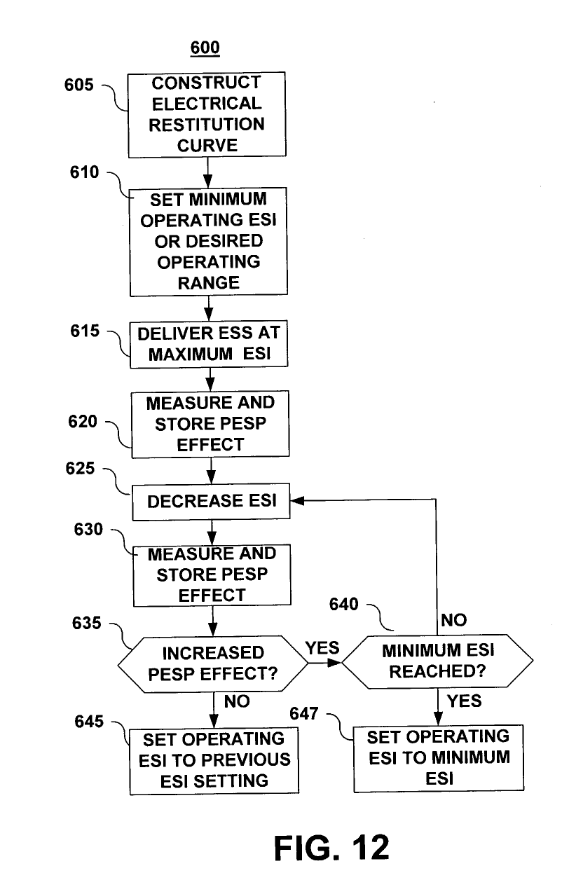

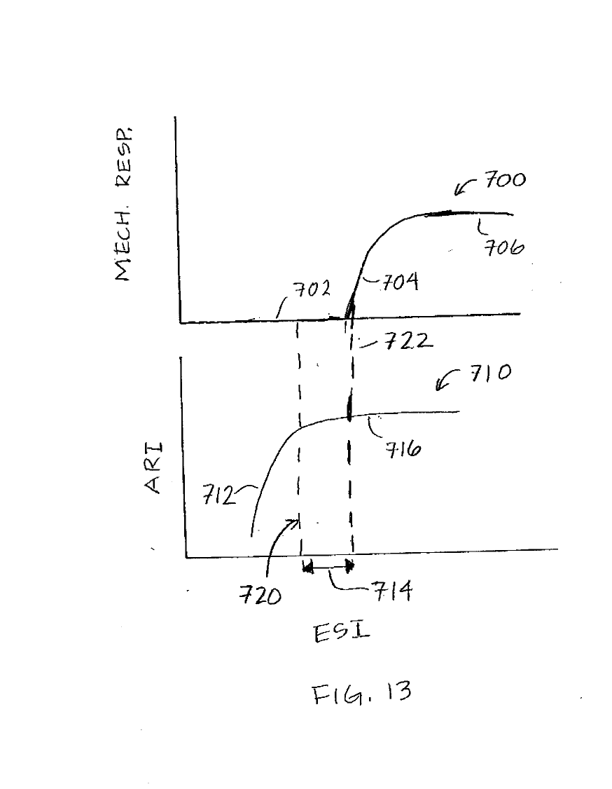

FIG. 12 is a flow chart summarizing a method for optimizing the ESI during ESS based on both electrical restitution and mechanical enhancement of post-extra systolic beats. At step 605, the electrical restitution curve is constructed, and a minimum operating ESI or operating ESI range is set corresponding to a desired point or range on the restitution curve at step 610 according to the methods described previously. At step 615, an iterative procedure begins for determining the ESI that meets the operating limits set forth at step 610 and results in maximum mechanical PESP effects.

At step 615, ESS is delivered at a maximum ESI limit set at step 610 or some predetermined maximum interval, which may be based on a percentage of or difference from a base rate interval associated with the intrinsic or paced heart rate. At step 620, the PESP effect is measured and stored. The PESP effect may be measured after a period of stabilization to allow the myocardial response to the adjusted ESI to reach a steady state. The PESP effect may be measured from a sensor capable of generating a signal proportional to myocardial contraction or wall motion or hemodynamic performance. Such sensors include, but are not limited to, a pressure sensor, a flow sensor, one or more single- or multi-axis accelerometers, a heart sound sensor, an impedance sensor, and so forth. Alternatively, a sensor indicative of metabolic state, such as an oxygen saturation sensor or pH sensor, may used to monitor the patient status during ESS. An index of hemodynamic or myocardial contractile performance or metabolic state is determined from the sensed signal acquired during post-extra systolic beats to determine the effectiveness of the extra systole in achieving mechanical PESP effects.

At step 625, the ESI is decreased by a predetermined decrement, and the PESP effect is measured and stored for the decreased ESI setting at step 630. If the PESP effect is greater than the PESP measured for the previous ESI setting, as determined at step 635 according to an improved hemodynamic, mechanical or metabolic parameter, method 600 continues to decrease the ESI setting in stepwise decrements by returning to step 625.

However, prior to returning to step 625, method 600 proceeds to decision step 640 to determine if the minimum ESI limit as set forth at step 610 based on the measurement of electrical restitution has been reached. If so, the operating ESI setting is set to the minimum ESI limit at step 647. If the minimum limit has not been reached, method 640 repeats steps 625 and 630 for decreasing the ESI and measuring the PESP effect until the PESP effect is no longer increasing with a shortening of the ESI, as determined at decision step 635. The operating ESI setting is adjusted to the previous ESI setting at which the PESP effect had reached a maximum. The iterative adjustments of ESI settings may alternatively be made in a random order or in a generally increasing order beginning from the minimum ESI limit until a maximal PESP effect is measured.

FIG. 13 is a graph of the mechanical response of the extra systole plotted versus ESI and a corresponding graph of the electrical restitution curve. The graph of mechanical response versus ESI is referred to herein as the “mechanical resitution curve” 700 as it represents the mechanical myocardial response to varying extra systolic intervals. The mechanical response plotted along the vertical axis may represent myocardial contractile force, myocardial shortening, cardiac chamber pressure development or dP/dt, or other measure of myocardial contraction strength or correlate thereof. Methods for measuring a mechanical restitution parameter that may be adapted for use in the present invention are generally disclosed in U.S. Pat. No. 6,438,408 issued to Mulligan et al., incorporated herein by reference in its entirety.

At very short ESIs, no mechanical response will occur and the mechanical restitution curve 700 is at a zero baseline 702. As the ESI is increased, the mechanical response is expected to increase suddenly at 704 until it reaches a maximum plateau 706 at relatively long ESIs, which produce a normal myocardial contraction.

In order to achieve a PESP effect, the mechanical contraction accompanying the electrical depolarization evoked by the extra systolic stimulation pulse must be absent or minimal. Therefore, a method for controlling the ESI setting based on achieving minimal or no mechanical response is contemplated. An operating ESI setting may be adjusted to a desired point on the mechanical restitution curve, for example the maximum ESI setting at which the mechanical response is still zero or at some point along the transition between a minimum and maximum mechanical response.

In one embodiment, the ESI is controlled based on both the mechanical restitution curve and the electrical restitution curve. By controlling the ESI based on both mechanical and electrical restitution, the operating ESI can be maintained at point that is not along the steep phase of the electrical restitution curve, which would otherwise cause undue risk of arrhythmias, nor along the maximal plateau of the mechanical restitution curve, which would otherwise preclude mechanical enhancement on post-extra systolic beats. By varying the ESI over a range of intervals and simultaneously measuring the mechanical response and the ARI interval on the extra systolic (S2) beats, the mechanical restitution curve 700 and the electrical restitution curve 710 may be constructed as illustrated in FIG. 13. A maximum ESI limit 722 is set based on the measurement of mechanical restitution, above which the mechanical S2 response is too large to achieve a PESP effect. A minimal ESI limit 720 is set based on the measurement of electrical restitution, below which the exaggerated shortening of the ARI elevates the risk of arrhythmia. The ESI range 714 bounded by the maximum ESI limit 722 and the minimum ESI limit 720 defines the desired operating range for the ESI during ESS. The operating ESI is therefore adjusted to an available setting within this range.

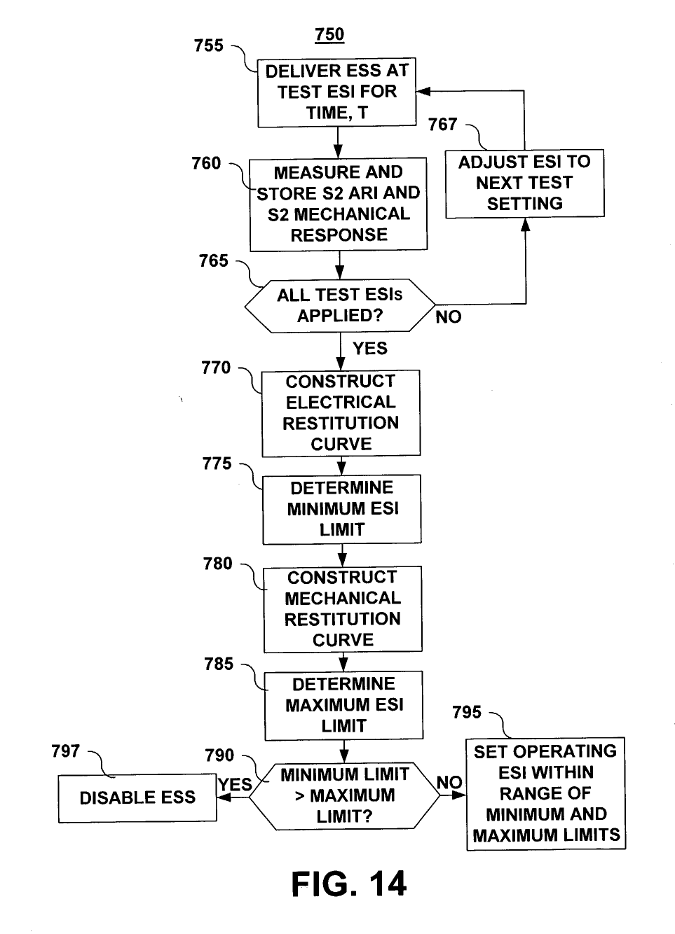

FIG. 14 is a flow chart summarizing steps included in a method for controlling the ESI during ESS based on electrical and mechanical restitution curves. At step 755, ESS is delivered at a test ESI for a period of time, T, to allow the steady state response of the myocardium to the ESI to be reached. At step 760, the ARI and the mechanical response of the extra systole S2 are measured and stored with the corresponding ESI label. The S2 ARI may be measured as described previously, during a single extra systole or as the average ARI measured from a number of extra systoles. The S2 mechanical response may likewise be measured on a single extra systole or as the average response measured from a number of extra systoles. The mechanical response is measured from a signal received from a sensor of myocardial contraction or hemodynamic function as described in conjunction with FIG. 13.

At step 765, method 750 determines if all test ESIs have been applied. A set of test ESIs may include two or more predetermined intervals or a number of intervals set relative to the sensed intrinsic heart rate or a cardiac pacing rate, for example as percentages of the intrinsic or paced rate interval. If all test intervals have not yet been applied, as determined at decision step 765, a new test interval is set at step 767. Steps 755 and 760 are repeated until all test intervals have been applied. Test intervals may be applied in a generally increasing, generally decreasing or random order.

After the S2 ARI and S2 mechanical response have been measured and stored for each test ESI, an electrical restitution curve is constructed at step 770, and a mechanical restitution curve is constructed at step 780. A minimum ESI limit is determined from the electrical restitution curve at step 775. The minimum ESI limit may be set as the shortest ESI occurring on the plateau phase of the electrical restitution curve, the transition point between the plateau phase and the steep phase, the peak or nadir of the biphasic “hump,” if present, or some other point on the restitution curve below which arrhythmia risk becomes undesirably high.

A maximum ESI limit is determined at step 785 from the mechanical restitution curve. The maximum ESI limit may be set as the longest ESI that results in no mechanical response (along the 0 baseline) or alternatively a point along the mechanical restitution curve which is associated with a degree of mechanical response to the extra systolic stimulus that is still expected to result in a PESP effect.

At step 790, the minimum ESI limit is compared to the maximum ESI limit. If the minimum limit is less than the maximum limit, the operating ESI is set at step 795 to an available ESI setting that is within the range of intervals bounded by the minimum and maximum limits. Mechanical restitution and electrical restitution curves may be re-constructed at any time to re-determine this optimal operating range.

If at any time, the minimum ESI limit set based on the electrical restitution curve is greater than the maximum ESI limit set based on the mechanical restitution curve, as determined at decision step 790, ESS is preferably disabled at step 797 to avoid increased arrhythmia risk. ESS may be re-enabled after subsequent mechanical and electrical restitution curve reconstructions determine a minimum ESI limit less than the maximum limit.

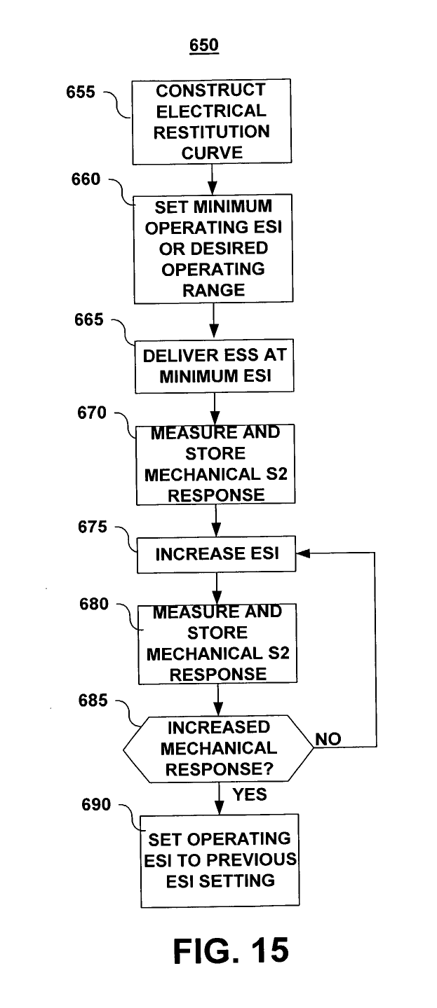

FIG. 15 is a flow chart summarizing steps included in an alternative method for controlling the ESI based on electrical restitution and mechanical restitution. Method 650 includes steps for setting a safe lower boundary or range for the ESI operating point based on electrical restitution measurements then optimizing the ESI within these bounds based on an iterative procedure for achieving a minimum mechanical response on the extra systolic beats.

At step 655, electrical restitution is measured, according to methods described previously, by constructing an electrical restitution curve from two or more points obtained by applying ESS at two or more ESIs and measuring the corresponding ARI from a sensed EGM (or subcutaneous ECG) signal, after an optional stabilization period. At step 660, a minimum ESI limit or ESI range is determined based on a desired operating point or range on the electrical restitution curve.

At step 665, ESS is delivered at the minimum ESI determined at step 660. The myocardial mechanical response to the extra systole is measured and stored at step 670 according to the methods described above. At step 675, the ESI is increased, and, after an optional period of stabilization, the mechanical response to the extra systole at the new ESI is measured and stored at step 680. The mechanical response to the new ESI is compared to the previously measured mechanical response at step 685. If the mechanical response is increased, the operating ESI is set, at step 690, to the previous ESI at which a lower myocardial mechanical response to the extra systole was measured. If the mechanical response is not increased, as determined at step 685, steps 675 and 680 are repeated until an increase in mechanical response is detected. An increase in mechanical response to the extra systole will weaken the mechanical PESP effect. Therefore method 650 allows the longest ESI to be identified that is greater than a safe minimum limit based on electrical restitution at which the mechanical response to the extra systole is constrained to a minimum.

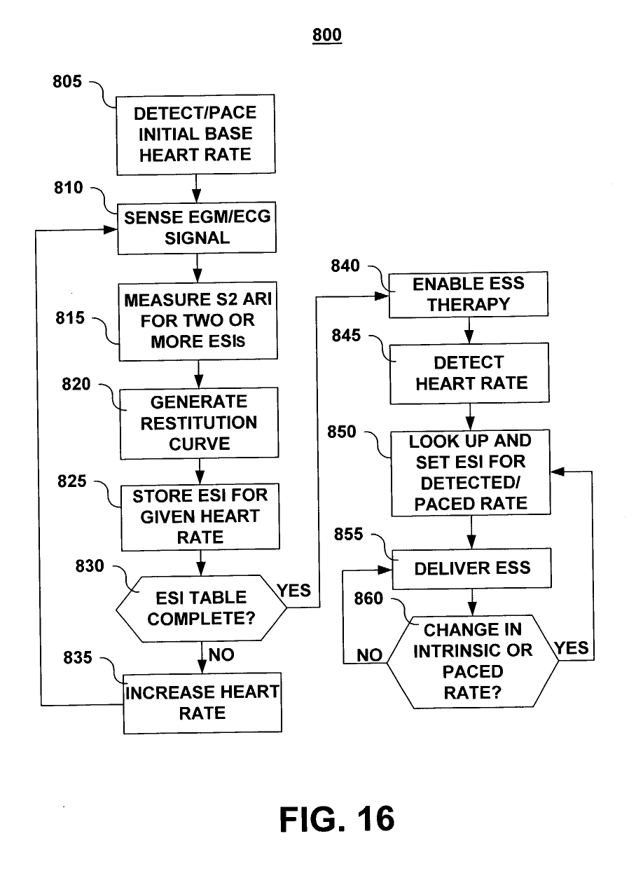

FIG. 16 is a flow chart summarizing a method for controlling ESS according to previously-determined ESIs based on electrical restitution measurements made over a range of heart rates. In this embodiment, electrical restitution curves are generated for a number of different heart rates such that an ESI may be determined from a restitution curve corresponding to a given heart rate or heart rate zone and stored for that heart rate. Automatic ESI adjustments may be made with variations in the intrinsic or paced heart rate without having to re-measure electrical restitution at the new heart rate. Method 800 shown in FIG. 16 includes steps for compiling a “look-up” table of ESIs and steps for delivering ESS at ESIs stored in the “look-up” table.

At step 805, the initial heart rate is determined. The heart rate may be an intrinsic, sinus rate or a paced rate. At step 810, an EGM/ECG signal selected for measuring extra systolic ARI is sensed, and the ARI is measured at step 815 according to methods described previously. The extra systolic ARI is measured for two or more ESIs such that an electrical restitution curve or defining slope may be generated at step 820.

At step 825, an ESI is determined based on a desired operating point on the restitution curve and stored for the given heart rate, or a pre-defined heart rate zone that includes the detected/paced heart rate. At step 830, method 800 determines if an ESI look-up table is complete for a number of heart rates or heart rate zones. If the table is not complete, the heart rate is increased at step 835. The steps performed for generating an ESI look-up table may be performed under clinical supervision such that heart rate increases at step 835 are exercise-induced, for example, by controlled treadmill or stationary bicycle exercise. A number of heart rate zones may be tested by asking the patient to exercise until the heart rate has reached a certain level, then asking the patient to maintain the same level of exertion while steps 810 through 825 are repeated for determining an appropriate ESI based on an electrical restitution curve for the given heart rate zone. Alternatively, heart rate increases may be controlled by increasing the primary base pacing rate in stepwise increments. Pacing induced increases in heart rate for generating an ESI look-up table may be performed automatically, with or without clinical supervision. Two or more heart rates or heart rate zones may be included in the ESI look-up table.

Once the look-up table is complete, ESS therapy is enabled at step 840. At step 845, the current heart rate, intrinsic or paced, is detected or identified, and the ESI is set at step 850 based on the value stored in the look-up table for the corresponding heart rate. ESS is delivered at step 855 at the ESI previously determined as the desired operating point on the electrical restitution curve for the given heart rate or heart rate zone. Throughout ESS delivery, the intrinsic/paced heart rate is monitored for shifts to different heart rate zones as indicated at decision step 860. If the intrinsic or paced rate increases or decreases to a different heart rate zone, method 800 returns to step 850 to adjust the ESI to the stored look-up table ESI value corresponding to the new heart rate zone. ESS is applied at the adjusted ESI at step 855.

Steps 805 through 835 for compiling an ESI look up table may be performed on a period basis, or upon detecting a change in restitution based on a sudden change in a measured ARI or other monitored restitution index, in order to update the stored ESIs according to changes in electrical restitution at varying heart rates that may occur with changes in disease state, medical therapy or other physiologic conditions.

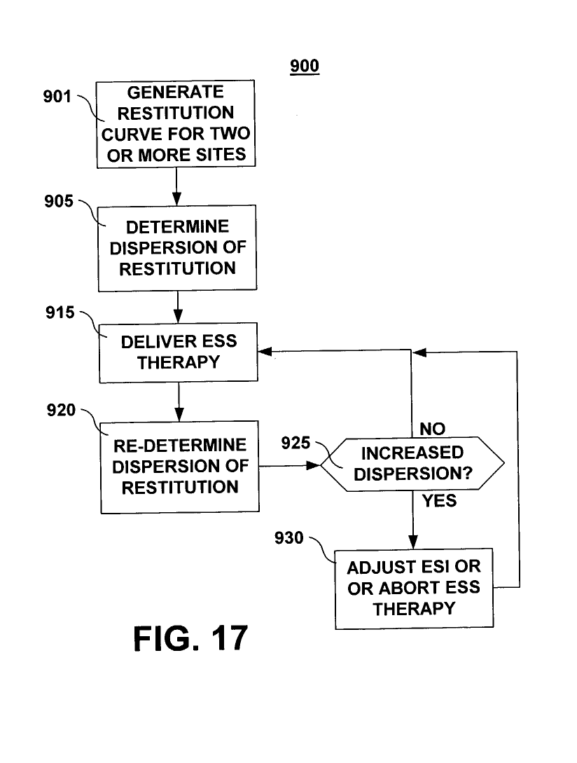

FIG. 17 is a flow chart summarizing steps included in a method for controlling ESS based on monitoring changes in the spatial dispersion of electrical restitution. Spatial dispersion of restitution refers to the difference in electrical restitution properties at different myocardial sites. An increase in the spatial dispersion of restitution properties, for example a greater difference between the slopes of restitution curves determined for two different myocardial sites, can lead to an increased heterogeneity of refractoriness and therefore a potentially increased risk of arrhythmias. Such increases are preferably avoidable and therefore if an increase in the spatial dispersion of electrical restitution is detected during ESS therapy, the ESS therapy is preferably either aborted or adjusted in a way to reduce the dispersion.

Method 900 begins at step 901 by generating electrical restitution curves for two or more myocardial sites. Electrical restitution curves are generated according to the methods described above by determining the extra systolic ARIs for two or more ESIs. ESS pulses may be delivered at one stimulation site at two or more ESIs, and restitution curves may be generated for the ESS site and/or other myocardial sensing sites. ESS pulses may alternatively be delivered at two or more sites with restitution curves generated for the stimulation sites and or other myocardial monitoring sites.

Restitution curves for measuring the spatial dispersion of restitution may be determined by measuring action potential duration related parameters from any available EGM and/or ECG sensing vectors. In one embodiment, restitution curves are generated based on ARIs measured from a right ventricular EGM signal and a left ventricular EGM signal such that dispersion of restitution between the right and left ventricles can be measured. Restitution curves may alternatively be generated from ARIs measured from one or more relatively global ECG or far-field EGM signals and one or more near-field EGM signals such that dispersion between restitution measured from a relatively local signal and restitution measured from a relatively global signal may be determined.

Spatial dispersion of restitution may be measured by determining the difference between a restitution index determined from restitution curves corresponding to two or more sensing sites. A restitution index may be a slope or other characteristic point or feature of the restitution curve. The dispersion of a restitution index is thus determined at step 905 as the greatest difference between R_{K}, R_{S}, or some other characteristic measure of the restitution curve for two or more EGM/ECG sensing vectors.

At step 915, ESS therapy is delivered according to any of the methods described above wherein the ESI is controlled based on a desired operating point on the restitution curve. During ESS, the dispersion of a restitution index is re-determined such that an increase in restitution dispersion may be detected at decision step 925.

In one embodiment, an index of restitution is measured on a beat-by-beat or less frequent basis by measuring the slope between the S1 ARI and the S2 ARI plotted versus the corresponding diastolic intervals as described previously in conjunction with the graphs of FIGS. 10A and 10B. In an alternative embodiment, a restitution curve slope may be determined by periodically measuring the ARI of the extra systole at the operating ESI and the ARI of an extra systole delivered at a test ESI, which may be an interval shorter or longer than the operating ESI. In yet other embodiments, a restitution curve for two or more sites (or sensing vectors) may be generated periodically by delivering an ESS pulse at varying ESIs, from which a restitution index and the spatial dispersion thereof may be determined.

As long as the restitution dispersion is not increased (decision step 925), ESS therapy delivery continues at step 915. If an increase in dispersion is detected, the ESI applied at one or more ESS sites may be adjusted or the ESS therapy may be aborted at step 930. If an adjustment to the ESI(s) is made at step 930, method 900 returns to step 915 to continue delivering ESS and monitoring for increased dispersion. If a number of attempts to adjust the ESI continue to result in increased restitution dispersion, ESS therapy may be aborted at step 930. Method 900 thereby allows ESS therapy to be delivered in a way that avoids increased spatial dispersion of electrical restitution, thereby avoiding increased risk of arrhythmias.

Thus, an implantable system and associated methods have been described for controlling an ESS therapy based on the electrical restitution properties of the myocardial tissue. The methods presented herein advantageously allow an operating ESI to be selected as a point on the electrical restitution curve which safely avoids an increased dispersion of refractoriness associated with heightened action potential duration shortening at relatively short ESIs. The methods presented herein further allow an optimal ESI to be selected based on electrical restitution for preventing an increased risk of arrhythmias and maximal PESP effects on the post-extra systolic beats for achieving the greatest hemodynamic benefit to the patient. Alternatively or additionally, the methods herein allow an optimal ESI to be selected based on electrical restitution and/or mechanical restitution wherein an operating ESI is selected safely above a minimum limit based on electrical restitution and/or below a maximum limit based on minimizing the mechanical response to the extra systole so as to maximize the PESP effect. Hence, the present invention allows the hemodynamic benefits of post-extra systolic potentiation to be gained in an electrical stimulation therapy for treating cardiac mechanical insufficiency while preventing an increased arrhythmia risk. While the present invention has been described according to specific embodiments presented herein, these embodiments are intended to be exemplary, not limiting, with regard to the following claims.

1. A method for providing extra systolic stimulation therapy, comprising:
sensing a signal associated with electrical activity of a heart;
measuring a parameter related to an action potential duration from the signal for at least two extra systolic intervals;
deriving a measure of electrical restitution from the measured action potential duration related parameter and a known extra systolic interval; and
adjusting an operating extra systolic interval used during delivery of extra systolic stimulation therapy based on the measure of electrical restitution.

2. A method for providing extra systolic stimulation therapy, comprising:
measuring myocyte action potential duration during one or more intrinsic extras systoles or one or more evoked extra-systoles for a plurality of extra systolic intervals;
measuring activation-recovery interval from an electrogram or an electrocardiogram for a plurality of cardiac cycles;
constructing an electrical restitution curve from the measured activation-recovery interval and the plurality of extra systolic intervals or a plurality of diastolic intervals; and
setting an initial operating extra systolic interval to a desired operating point on the constructed electrical restitution curve or at a transition between a steep phase and a plateau phase of the electrical restitution curve.

3. An apparatus for providing extra systolic stimulation therapy, comprising:
means for sensing a signal associated with electrical activity of a heart;
means for measuring a parameter related to an action potential duration from the signal for at least two extra systolic intervals;
means for deriving a measure of electrical restitution from the measured action potential duration related parameter and a known extra systolic interval; and
means for adjusting an operating extra systolic interval used during delivery of extra systolic stimulation therapy based on the measure of electrical restitution.

4. An apparatus for providing extra systolic stimulation therapy, comprising:
means for measuring myocyte action potential duration during one or more intrinsic extras systoles or one or more evoked extra-systoles for a plurality of extra systolic intervals;
means measuring activation-recovery interval from an electrogram or an electrocardiogram for a plurality of cardiac cycles;
means for constructing an electrical restitution curve from the measured activation-recovery interval and the plurality of extra systolic intervals or a plurality of diastolic intervals; and
means for setting an initial operating extra systolic interval to a desired operating point on the constructed electrical restitution curve or at a transition between a steep phase and a plateau phase of the electrical restitution curve.

5. A computer readable medium for causing a programmable processor to perform a method of delivering extra systolic stimulation, comprising:
instructions for measuring myocyte action potential duration during one or more intrinsic extras systoles or one or more evoked extra-systoles for a plurality of extra systolic intervals;
instructions for measuring an activation-recovery interval from an electrogram or an electrocardiogram for a plurality of cardiac cycles;
instructions for constructing an electrical restitution curve from the measured activation-recovery interval and the plurality of extra systolic intervals or a plurality of diastolic intervals; and
instructions for setting an initial operating extra systolic intervals to a desired operating point on the constructed electrical restitution curve or at the transition between a steep phase and a plateau phase of the electrical restitution curve.

6. A computer readable medium for causing a programmable processor to perform a method of delivering extra systolic stimulation, comprising:
instructions for sensing a signal associated with electrical activity of a heart;
instructions for measuring a parameter related to an action potential duration from the signal for at least two extra systolic intervals;
instructions for deriving a measure of electrical restitution from the measured action potential duration related parameter and a known extra systolic interval; and
instructions for adjusting an operating extra systolic interval used during delivery of an extra systolic stimulation therapy based on the measure of the electrical restitution.
